(19) United States
(12) Patent Application Publication
(10) Pub. No.: US 2018/0246121 A1
(43) Pub. Date: Aug. 30, 2018

(54) IMMUNOLOGICAL TEST METHOD AND IMMUNOLOGICAL TEST KIT

(71) Applicants: Fuso Parmaceutical Industries, Ltd., Osaka (JP); Canon Kabushiki Kaisha, Ohta-ku (JP)

(72) Inventors: Masahiro Asakura, Osaka-shi (JP); Aya Keshi, Osaka-shi (JP); Masaaki Kobayashi, Ohta-ku (JP)

(73) Assignees: Fuso Pharmaceutical Industries, Ltd., Osaka (JP); Canon Kabushiki Kaisha, Ohta-ku (JP)

(21) Appl. No.: 15/753,368

(22) PCT Filed: Aug. 19, 2016
(86) PCT No.: PCT/JP2016/074181

§ 371 Date: Feb. 19, 2018

(30) Foreign Application Priority Data
Aug. 21, 2015 (JP) 2015-163839

(51) Int. Cl. G01N 33/68; C07K 16/24; G01N 33/543; C07K 7/08

(52) U.S. Cl. CPC G01N 33/6863; C07K 7/08; G01N 33/54393; C07K 16/24

(57) ABSTRACT

It was found that when measuring HMGB1 in samples using antibodies that bind to both HMGB2 and HMGB1, the reaction between HMGB2 and HMGB1 antibodies can be suppressed by coexisting HMGB2 absorbents (HMGB2 antibodies and/or HMGB2-derived peptides that inhibit the binding between HMGB2 and HMGB1 antibodies). More specifically, it was revealed that HMGB1 alone in samples can be measured or detected by contacting the samples with HMGB1 antibodies in the presence of HMGB2 absorbents.

### TECHNICAL FIELD

The present invention relates to methods of measuring or detecting High Mobility Group Box 1 (hereinafter, sometimes abbreviated as “HMGB1”) which is a possible disease marker for sepsis and the like, and kits and such for measuring or detecting HMGB1.

### BACKGROUND ART

High Mobility Group Proteins (hereinafter, “HMGs”) are non-histone proteins included in chromatin structure, and are proteins commonly included in many higher plants and animals. There are several types of HMGs and examples include HMGB1, HMGB2, HMGB3, HMGB8, and HMGB17. These HMGs characteristically have high amino-acid-sequence homology to each other. For example, the homology of a human-derived HMGB2 to a human-derived HMGB1 is 80% or higher.

In 1999, Wang et al. (Non-patent Document 1) showed that HMGB1 may be a marker for sepsis, and HMGB1 has come to draw attention as a mediator expressed in the late stage of septic shock. Furthermore, since HMGB1 is released to the outside of the nucleus at the onset of sepsis and exhibits severe cytotoxicity, its possible involvement in lethality during sepsis has been pointed out.

On the other hand, there is also a report (Non-patent Document 2) on the release of HMGB2 into blood. Maruyama et al. have confirmed that HMGB2 is released into the serum of ulcerative colitis patients through Western blotting. This means that, in human blood, HMGB1 and HMGB2 may be detected simultaneously. Therefore, for discrimination of sepsis, methods and reagents for specifically measuring HMGB1, even in the presence of HMGB2, are needed.

Japanese Patent No. 5055598 (Patent Document 1) includes a description relating to acquisition of antibodies capable of discriminating between HMGB1 and HMGB2, as a means for specifically measuring HMGB1 in samples. Specifically, first, a peptide comprising the amino acid sequence of HMGB1 was used as an immunogen to immunize animals, and HMGB1 antibody production was prompted. However, as described above, the amino acid sequence homology between HMGB1 and HMGB2 is very high. Because of this, in practice, HMGB1 antibodies that bind not only to HMGB1 but also to HMGB2 were produced in large numbers. Therefore, after antibody production, HMGB1 antibodies that also bind to HMGB2 were removed by affinity chromatography that uses HMGB2 as the ligand, and HMGB1 antibodies that bind specifically to HMGB1 were obtained.

### PRIOR ART DOCUMENTS

### Patent Documents

[Patent Document 1] Japanese Patent No. 5055598
[Patent Document 2] Japanese Patent Application Publication No. (JP-A) 2013-122402 (unexamined, published Japanese Patent Application) Analyzing Device for Specimen Inspection (Canon Inc., Canon Chemicals Inc.)

### Non-Patent Documents

[Non-patent Document 1] SCIENCE, 285, 248-251, 1999
[Non-patent Document 2] Clinical Chemistry, 49, 9, 1535-1537, 2003
[Non-patent Document 3] Rockland Immunochemicals, Inc. homepage, [online], [Jun. 1, 2015 retrieved], Internet <URL: http://www.rockland-inc.com/uploadedFiles/Support/Protocols/Peptide-Competition-Protocol.pdf>
[Non-patent Document 4] Intensive Care Medicine, 33, 1347-1353, 2007
[Non-patent Document 5] Blood Purification, 32, 139-142, 2011

### SUMMARY OF THE INVENTION

### Problems to be Solved by the Invention

As described above, specific measurement of HMGB1 is required for the discrimination of sepsis even in the presence of HMGB2. However, since the amino acid sequence homology between HMGB1 and HMGB2 is very high, when HMGB1 antibodies are produced in immunized animals, HMGB1 antibodies that bind not only to HMGB1 but also to HMGB2 are produced in large numbers.

Affinity chromatography method is known as a means for obtaining HMGB1 antibodies that bind only to HMGB1 from the produced antibodies, as mentioned in the above-described Japanese Patent No. 5055598. However, this method not only increases the number of steps but also decreases the recovered amount of the desired substance due to purification; therefore, there is the problem of increase in production cost.

This way, while establishing a means for specifically measuring HMGB1 is in demand, obtaining HMGB1 antibodies that specifically bind to HMGB1 is not easy. The present invention was achieved in view of such circumstances, and an objective is to provide methods of selectively measuring or detecting HMGB1 using HMGB1 antibodies, and kits and the like for use in such methods.

### Means for Solving the Problems

The present inventors carried out dedicated research to solve the above-mentioned problems, and as a result discovered that when measuring HMGB1 in samples using HMGB1 antibodies that also bind to HMGB2, coexistence of HMGB2 absorbents can suppress binding between HMGB2 and the HMGB1 antibodies, and can serve to specifically detect HMGB1.

The present invention is based on such finding, and relates to methods of measuring or detecting HMGB1 in samples using HMGB1 antibodies, which comprise contacting samples with HMGB1 antibodies in the presence of HMGB2 absorbents.

Furthermore, the present invention relates to kits or reagents for measuring or detecting HMGB1 contained in samples using HMGB1 antibodies, which comprise at least an HMGB1 antibody and an HMGB2 absorbent, and which are used by contacting the samples with the HMGB1 antibody in the presence of the HMGB2 absorbent.

In the methods of the present invention, binding between HMGB2 and an HMGB1 antibody can be suppressed by allowing an HMGB1 antibody and an HMGB2 absorbent to coexist in a sample. Therefore, even when both HMGB1 and HMGB2 are included in samples, HMGB1 can be measured accurately.

Furthermore, in the methods of the present invention, HMGB1 antibodies having affinity also to HMGB2 can be used. Accordingly, many of the HMGB1 antibodies, which are easily obtained by commonly known antibody production techniques that use immunized animals, can be used in the methods of the present invention.

Furthermore, in the methods of the present invention, HMGB2 antibodies can be used as the HMGB2 absorbent. HMGB2 antibodies can be obtained from generally known techniques for antibody production using immunized animals; therefore, HMGB2 absorbents can be prepared easily.

Furthermore, in the methods of the present invention, a peptide comprising the HMGB2-derived amino acid sequence of formula (I) or (II) may be used as the HMGB2 absorbent. Since these peptides can be synthesized easily, HMGB2 absorbents can be prepared at a lower cost.

| | | |
|---|---|---|
| (I): | MGKGDPNKPRGKMSSYA | (SEQ ID NO: 3) |
| (II): | CREEHKKKHPDSSVNFAEFS | (SEQ ID NO: 4) |

Furthermore, regarding the kits and reagents of the present invention, at least an HMGB2 absorbent is comprised therein, and binding between HMGB2 and HMGB1 antibodies can be suppressed simply by mixing the HMGB2 absorbent with samples upon measurement. Therefore, HMGB1 can be measured accurately with simple kit and reagent formulations.

Furthermore, in the kits and reagents of the present invention, HMGB1 antibodies having affinity also to HMGB2 can be used. Therefore, it becomes possible to use many HMGB1 antibodies obtained by generally known techniques for producing antibodies using immunized animals, and kits and reagents can be prepared easily.

Furthermore, in the kits and reagents of the present invention, HMGB2 antibodies can be used as the HMGB2 absorbent. HMGB2 antibodies can be obtained by generally known techniques for antibody production that use immunized animals; therefore, HMGB2 absorbents can be prepared easily.

Furthermore, in the kits and reagents of the present invention, a peptide comprising the HMGB2-specific amino acids of formula (I) or (II) may be used as the HMGB2 absorbent. Since these peptides can be synthesized easily, HMGB2 absorbents can be prepared at a lower cost.

| | | |
|---|---|---|
| (I): | MGKGDPNKPRGKMSSYA | (SEQ ID NO: 3) |
| (II): | CREEHKKKHPDSSVNFAEFS | (SEQ ID NO: 4) |

More specifically, the present invention relates to the following [1] to [17]:

[1] a method of measuring or detecting HMGB1 in a sample, which comprises the step of contacting an HMGB1 antibody with the sample in the presence of an HMGB2 absorbent;
[2] the method of [1], wherein the HMGB1 antibody is an antibody having affinity to both HMGB1 and HMGB2;
[3] the method of [1] or [2], wherein the HMGB2 absorbent comprises at least an HMGB2 antibody;
[4] the method of [1] or [2], wherein the HMGB2 absorbent comprises at least a peptide comprising the amino acid sequence of the following formula (I) or (II):

| | | |
|---|---|---|
| (I): | MGKGDPNKPRGKMSSYA | (SEQ ID NO: 3) |
| or | | |
| (II): | CREEHKKKHPDSSVNFAEFS; | (SEQ ID NO: 4) |

[5] the method of any one of [1] to [4], which comprises measuring or detecting HMGB1 by an immunological test method;
[6] a kit for measuring or detecting HMGB1, which comprises at least a first reagent comprising an HMGB1 antibody and a second reagent comprising an HMGB2 absorbent;
[7] the kit of [6], wherein the HMGB1 antibody is an antibody having affinity to both HMGB1 and HMGB2;
[8] the kit of [6] or [7], wherein the HMGB2 absorbent comprises at least an HMGB2 antibody;
[9] the kit of [6] or [7], wherein the HMGB2 absorbent comprises at least a peptide comprising the amino acid sequence of the following formula (I) or (II):

| | | |
|---|---|---|
| (I): | MGKGDPNKPRGKMSSYA; | (SEQ ID NO: 3) |
| or | | |
| (II): | CREEHKKKHPDSSVNFAEFS; | (SEQ ID NO: 4) |

[10] the kit of any one of [6] to [9], which is for use in measuring or detecting HMGB1 using an immunological test method;
[11] a reagent for measuring or detecting HMGB1, which comprises at least an HMGB1 antibody and an HMGB2 absorbent;
[12] the reagent of [11], wherein the HMGB1 antibody is an antibody having affinity to both HMGB1 and HMGB2;
[13] the reagent of [11] or [12], wherein the HMGB2 absorbent comprises at least an HMGB2 antibody;
[14] the reagent of [11] or [12], wherein the HMGB2 absorbent comprises at least a peptide comprising the amino acid sequence of the following formula (I) or (II):

| | | |
|---|---|---|
| (I): | MGKGDPNKPRGKMSSYA; | (SEQ ID NO: 3) |
| or | | |
| (II): | CREEHKKKHPDSSVNFAEFS; | (SEQ ID NO: 4) |

[15] the reagent of any one of [11] to [14], which is for use in measuring or detecting HMGB1 using an immunological test method;
[16] a peptide comprising the amino acid sequence of the following formula (I) or (II):

| | | |
|---|---|---|
| (I): | MGKGDPNKPRGKMSSYA; | (SEQ ID NO: 3) |
| or | | |
| (II): | CREEHKKKHPDSSVNFAEFS, | (SEQ ID NO: 4) |

wherein the peptide inhibits binding between HMGB2 and an HMGB1 antibody; and
[17] a sample which comprises the peptide of [16].

### Effects of the Invention

The present invention provides methods of specifically measuring or detecting HMGB1 in samples, and kits and reagents for specifically measuring or detecting HMGB1 in samples. The methods, kits, and reagents of the present invention are characterized by serving to contact an HMGB1 antibody with a sample in the presence of an HMGB2 absorbent (an HMGB2 antibody and/or an HMGB2-derived peptide that inhibits binding between HMGB2 and the HMGB1 antibody). In the present invention, it is possible to use HMGB1 antibodies having affinity also to HMGB2, rather than HMGB1-specific antibodies that are difficult to obtain. Such antibodies can be obtained easily by generally known techniques for antibody production using immunized animals. Therefore, use of the methods, kits, and reagents of the present invention enables easy and simple selective detection of HMGB1 in samples.

Furthermore, among the substances that constitute HMGB2 absorbents of the present invention, HMGB2 antibodies and the HMGB2-derived peptide (II) (CREEHKKKHPDSSVNFAEFS/SEQ ID NO: 4) have effects of promoting or increasing binding between HMGB1 antibodies and HMGB1 in addition to effects of inhibiting binding between HMGB2 and HMGB1 antibodies or effects of competing with binding between HMGB2 and HMGB1 antibodies. Therefore, when HMGB2 antibodies or the HMGB2-derived peptide (II) are used as the HMGB2 absorbent, HMGB1 in samples can be selectively detected with higher efficiency.

### BRIEF DESCRIPTION OF THE DRAWINGS

FIG. 1 is a graph showing non-specific reactions by HMGB2 and suppression of the reactions by anti-HMGB2 antibodies in HMGB1-ELISA.

FIG. 2 is a graph showing effects of anti-HMGB2 antibodies on HMGB1-ELISA.

FIG. 3 is a graph showing HMGB2 measurements and absorption by antibodies in HMGB1-ELISA.

FIG. 4A is a graph showing effects of the absorbent (HMGB2 antibody No. 3) on an HMGB1/2-mixed sample.

FIG. 4B is a graph showing effects of the absorbent on an HMGB1/2-mixed sample in the low-concentration range.

FIG. 5 is a graph showing the effects of the absorbent (MHGB2 antibody) on an HMGB1 and HMGB1/2 mixture.

FIG. 6 shows graphs indicating the absorption effects by an anti-HMGB2 antibody in ELISA using HMGB1 polyclonal antibody No. 12. A: HMGB-1 (50 ng/mL) was measured using a labeled HMGB1 polyclonal antibody (No. 12). B: HMGB-2 (1,820 ng/mL) was measured using a labeled HMGB1 polyclonal antibody (No. 12).

FIG. 7 shows graphs indicating the absorption effects by an anti-HMGB2 antibody in ELISA using HMGB1 polyclonal antibody No. 14. A: HMGB-1 (50 ng/mL) was measured using a labeled HMGB1 polyclonal antibody (No. 14). B: HMGB-2 (1,820 ng/mL) was measured using a labeled HMGB1 polyclonal antibody (No. 14).

FIG. 8 shows graphs indicating the effects of peptide MH2-1 on measurements of HMGB1 and HMGB2. A: HMGB1 12.5 ng/mL. B: HMGB2 1,820 ng/mL.

FIG. 9 shows graphs indicating the effects of peptide MH2-2 on measurements of HMGB1 and HMGB2. A: HMGB1 12.5 ng/mL. B: HMGB2 1,820 ng/mL.

FIG. 10 shows graphs indicating the effects of HMGB2 peptides in ELISA using HMGB1 polyclonal antibody No. 12. A: HMGB-1 (50 ng/mL) was measured using a labeled HMGB1 polyclonal antibody (No. 12). B: HMGB-2 (1,820 ng/mL) was measured using a labeled HMGB1 polyclonal antibody (No. 12).

FIG. 11 shows graphs indicating the effects of HMGB2 peptides in ELISA using HMGB1 polyclonal antibody No. 14. A: HMGB-1 (50 ng/mL) was measured using a labeled HMGB1 polyclonal antibody (No. 14). B: HMGB-2 (1,820 ng/mL) was measured using a labeled HMGB1 polyclonal antibody (No. 14).

FIG. 12 shows graphs indicating the effects of HMGB2 peptides in ELISA using HMGB1 monoclonal antibody HMa176. A: HMGB-1 (50 ng/mL) was measured using labeled HMGB1 antibody HMa176. B: HMGB-2 (1,820 ng/mL) was measured using labeled HMGB1 antibody HMa176.

FIG. 13 shows the amino acid sequence alignment of human HMGB1 (SEQ ID NO: 1/NCBI Accession No. CAG33144.1) and human HMGB2 (SEQ ID NO: 2/NCBI Accession No. AAI00020.1)

FIG. 14 shows inhibition of HMGB2-non-specific reactions by an HMGB2 antibody or an HMGB2 peptide. A) An HMGB1 antibody weakly binds to highly homologous HMGB2. B) Addition of an HMGB2 antibody blocks binding of the HMGB1 antibody (antibody A) to HMGB2, and HMGB1 is measured specifically. Since the HMGB2 antibody has a large molecular weight and readily causes steric hinderance, it may also block an antibody (antibody B). C) Addition of an HMGB2 peptide neutralizes binding of HMGB1 antibody A to HMGB2, and HMGB1 is measured specifically. However, the peptide molecular weight is small, and neutralization reaction by the peptide does not take place for HMGB1 antibody B which binds to a different epitope.

FIG. 15 shows the three-dimensional structural model and the DNA-binding site of HMGB1. The DNA-binding site at the N-terminal-side of HMGB1 is shown. An acidic substance such as DNA is considered to bind to the region surrounded by H1 to H3 via basic amino acids such as lysine included in the helical structures of H1 to H3.

FIG. 16 shows HMGB1 and the DNA-binding site. A) Negatively-charged substances such as DNA and heparin bind to regions of HMGB1 rich in basic amino acids such as lysine. Therefore, due to steric hinderance, an antibody cannot bind sufficiently. B) Since HM2 peptide binds to negatively-charged substances such as DNA and heparin, and inhibits binding to HMGB1, inhibition of antibody binding due to steric hinderance hardly takes place.

### MODE FOR CARRYING OUT THE INVENTION

Hereinbelow, the present invention will be described in detail. Embodiments indicated below are mere examples, and the present invention is not necessarily limited to the following.

The present invention relates to methods of measuring or detecting HMGB1 in samples, which comprises the step of contacting an HMGB1 antibody with the samples in the presence of an HMGB2 absorbent.

In the present invention, samples refer to solutions which contain or are suspected to contain HMGB1. Examples include isolated biological samples such as blood, serum, plasma, urine, semen, spinal fluid, saliva, sweat, tear, ascites, and amniotic fluid derived from humans or other animals (for example, mice, rats, rabbits, dogs, cats, bovines, horses, pigs, goats, sheep, monkeys [for example, rhesus monkeys and cynomolgus monkeys], chimpanzees, chickens, and zebrafish), and diluted solutions containing them, but are not limited thereto. Furthermore, solutions obtained by mixing solutions which contain or are suspected to contain HMGB1 with buffers and such are also included in the samples of the present invention. Methods of obtaining such samples are well known to those skilled in the art. Various aqueous solvents can be used as the solvents for mixing or dilution. Examples include purified water, physiological saline solution, or various buffers such as Tris buffer, phosphate buffer, or phosphate buffered saline solution, but are not limited thereto. Furthermore, the pH of the buffers is not particularly limited, and suitable pH can be appropriately selected. Generally, pH in the range of pH 3 to 12 can be selected and used, but is not limited thereto. Furthermore, for structural protection of substances to be measured, the solvents may appropriately contain one, two or more kinds of proteins such as bovine serum albumin (BSA), human serum albumin (HSA), and casein; various saccharides; powdered skimmed milk; various animal sera such as normal rabbit serum; various antiseptics such as sodium azide and antibiotics; and various surfactants such as nonionic surfactants, amphoteric surfactants, and anionic surfactants.

The origin of HMGB1 is not limited in the present invention. In the present invention, without being limited to the following, HMGB1 derived from humans, mice, rats, rabbits, dogs, cats, bovines, horses, pigs, goats, rhesus monkeys, cynomolgus monkeys, chimpanzees, chickens, zebrafish, or such can be measured or detected.

The NCBI (National Center for Biotechnology Information, U.S. National Library of Medicine) database accession numbers and the SEQ ID NOs used herein are shown below for each of the HMGB1 amino acid sequences:

human [Homo sapiens] CAG33144.1/SEQ ID NO: 1

mouse [Mus musculus] AAI10668.1/SEQ ID NO: 7

rat [Rattus norvegicus] NP_037095.1/SEQ ID NO: 8

rabbit [Oryctolagus cuniculus] NP_001164752.1/SEQ ID NO: 9

dog [Canis lupus familiaris] AAN11319.1/SEQ ID NO: 10

cat [Felis catus] XP_006927254.1/SEQ ID NO: 11

bovine [Bos taurus] AA102930.1/SEQ ID NO: 12

horse [Equus caballus] BAF33339.1/SEQ ID NO: 13

pig [Sus scrofa] NP_001004034.1/SEQ ID NO: 14

goat [Capra hircus] XP_005687595.1/SEQ ID NO: 15

rhesus monkey [Macaca mulatta] AFJ72047.1/SEQ ID NO: 16

cynomolgus monkey [Macaca fascicularis] NP_001270285.1/SEQ ID NO: 17

chimpanzee [Pan troglodytes] XP_509611.1/SEQ ID NO: 18

chicken [Gallus gallus] NP_990233.1/SEQ ID NO: 19

zebrafish [Danio rerio] AAH67193.1/SEQ ID NO: 20

HMGB1 antibodies of the present invention are not particularly limited as long as they have binding activity to HMGB1. While HMGB1 antibodies that specifically bind to HMGB1 as well as HMGB1 antibodies that have affinity to both HMGB1 and HMGB2 can be used, the latter antibodies are suitably used in the present invention.

Furthermore, in the present invention, the types, origins, and such of the HMGB1 antibodies are also not limited. For example, antibodies obtained from various immunized animals such as mice, rabbits, and goats can be used. Polyclonal antibodies, anti-serum comprising polyclonal antibodies, monoclonal antibodies, or antibody fragments thereof (for example, Fab, F(ab′)2, and Fab′), and low-molecular-weight antibodies (for example, scFv (single-chain Fv), diabody, and sc(Fv)2 (single-chain (Fv)2), and multimers thereof (for example, dimers, trimers, tetramers, and polymers) can also be used.

For example, polyclonal antibodies can be prepared by immunizing animals such as rabbits with purified HMGB1 or partial peptides thereof, collecting blood after a certain period of time, and then removing blood clots. In addition, monoclonal antibodies can be prepared by fusing bone tumor cells with antibody-producing cells from animals immunized with HMGB1 or partial peptides thereof, isolating cells from a single clone that produces the antibody of interest (hybridoma), and obtaining antibodies from the cells.

Antibody fragments can be produced by digesting antibodies with enzymes. Papain, pepsin, plasmin, and such are known examples of enzymes that produce antibody fragments. Alternatively, DNAs encoding such antibody fragments can be constructed, and after introducing them into expression vectors, the antibody fragments can be expressed in appropriate host cells.

An scFv can be obtained by linking VH and VL of an antibody. In an scFv, VH and VL are linked via a linker, preferably via a peptide linker. The peptide linkers which link the V regions are not particularly limited. For example, any single-chain peptide consisting of approximately 3 to 25 residues can be used as the linker. A diabody is a dimer composed of two scFvs. An sc(Fv)2 is a low-molecular-weight antibody in which two VHs and two VLs are linked by linkers or such to produce a single chain. An sc(Fv)2 can be prepared, for example, by linking two scFvs with a linker.

HMGB1 antibodies of the present invention can be, for example, antibodies that recognize regions comprising HMGB1-derived amino acid sequences with low homology to the amino acid sequence of HMGB2. The regions comprising HMGB1-derived amino acid sequences with low homology to the amino acid sequence of HMGB2 can be rephrased as amino acid sequence regions that are not shared by HMGB1 and HMGB2 and comprise HMGB1-derived amino acid sequences, or amino acid sequence regions whose three-dimensional structures are different between HMGB1 and HMGB2 and which comprise HMGB1-derived amino acid sequences. Examples of such regions include, but are not limited to, regions comprising the HMGB1-specific amino acid sequence of formula (I′) or (II′).

(I′): MGKGDPKKPRGKMSSYA (SEQ ID NO: 5/the 1st to 17th amino acids in the amino acid sequence of the full-length human HMGB1 (SEQ ID NO: 1))

(II′): CREEHKKKHPDASVNFSEFS (SEQ ID NO: 6/the 23rd to 42nd amino acids in the amino acid sequence of the full-length human HMGB1 (SEQ ID NO: 1))

In the present invention, use of two types of HMGB1 antibodies is also possible. In that case, the two types of antibodies may be both HMGB1-specific antibodies or may be HMGB1 antibodies having affinity to both HMGB1 and HMGB2. Alternatively, in the present invention, an HMGB1-specific antibody and an HMGB1 antibody having affinity to both HMGB1 and HMGB2 may be used in combination. However, in the present invention, it is preferred that the two types of antibodies are both antibodies having affinity to both HMGB1 and HMGB2. For example, in the later-described ELISA methods, both immobilized antibody and enzyme-labeled antibody are preferably HMGB1 antibodies having affinity to both HMGB1 and HMGB2.

Furthermore, there is no limitation to the strength of affinity to HMGB1 and affinity to HMGB2 of HMGB1 antibodies having affinity to both HMGB1 and HMGB2. For example, affinity to HMGB1 and affinity to HMGB2 may be at the same level, or the antibody may bind more strongly to HMGB1 than to HMGB2. Alternatively, among the two types of HMGB1 antibodies, one may be an HMGB1 antibody whose affinity to HMGB1 and affinity to HMGB2 are at the same level, and the other may be an HMGB1 antibody that binds more strongly to HMGB1 than to HMGB2.

In the present invention, HMGB2 absorbents refer to substances that inhibit binding between HMGB2 and HMGB1 antibodies, or substances that compete with binding between HMGB2 and HMGB1 antibodies.

The absorbents of the present invention are not particularly limited as long as they have an activity of inhibiting binding between HMGB2 and HMGB1 antibodies or an activity of competing with binding between HMGB2 and HMGB1 antibodies (hereinafter referred to as activities of inhibiting binding between HMGB2 and HMGB1 antibodies), but the following can be presented as examples.
HMGB2 antibodies
HMGB2-derived peptides comprising or consisting of the amino acid sequence of formula (I) or (II):
   (I): MGKGDPNKPRGKMSSYA (SEQ ID NO: 3/the 1st to 17th amino acids in the amino acid sequence of the full-length human HMGB2 (SEQ ID NO: 2))
   (II): CREEHKKKHPDSSVNFAEFS (SEQ ID NO: 4/the 23rd to 42nd amino acids in the amino acid sequence of the full-length human HMGB2 (SEQ ID NO: 2))
HMGB2-derived peptides having activities of inhibiting binding between HMGB2 and HMGB1 antibodies, which consist of an amino acid sequence having substitution, deletion, addition and/or insertion of one or more (for example, two, three, four, five, or ten) amino acids in the amino acid sequence of formula (I) or (II).

Absorbents in the present invention can comprise one or more of these antibodies or peptides.

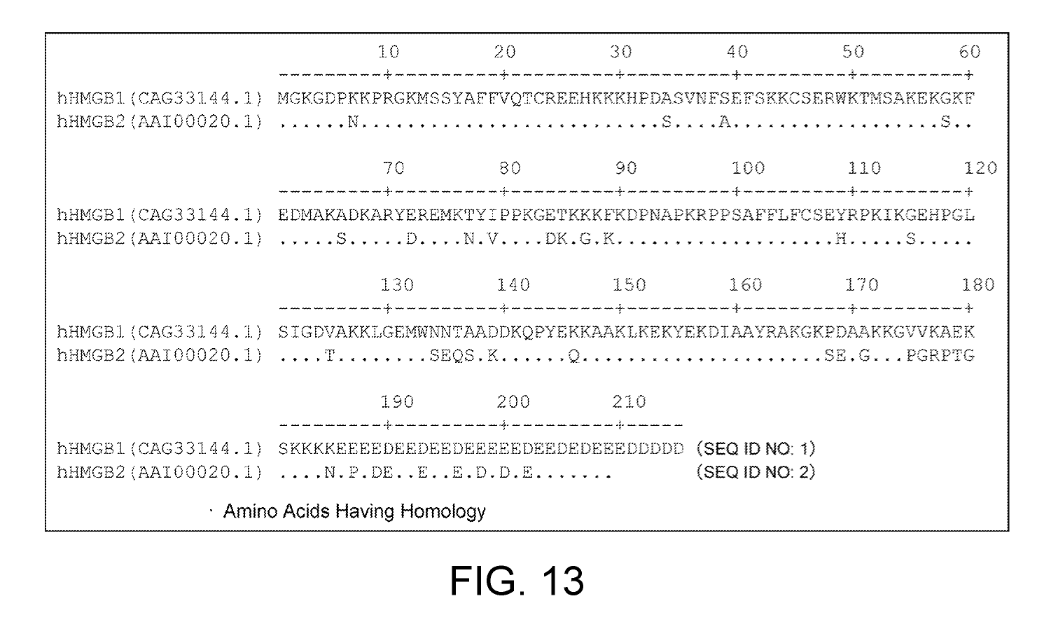

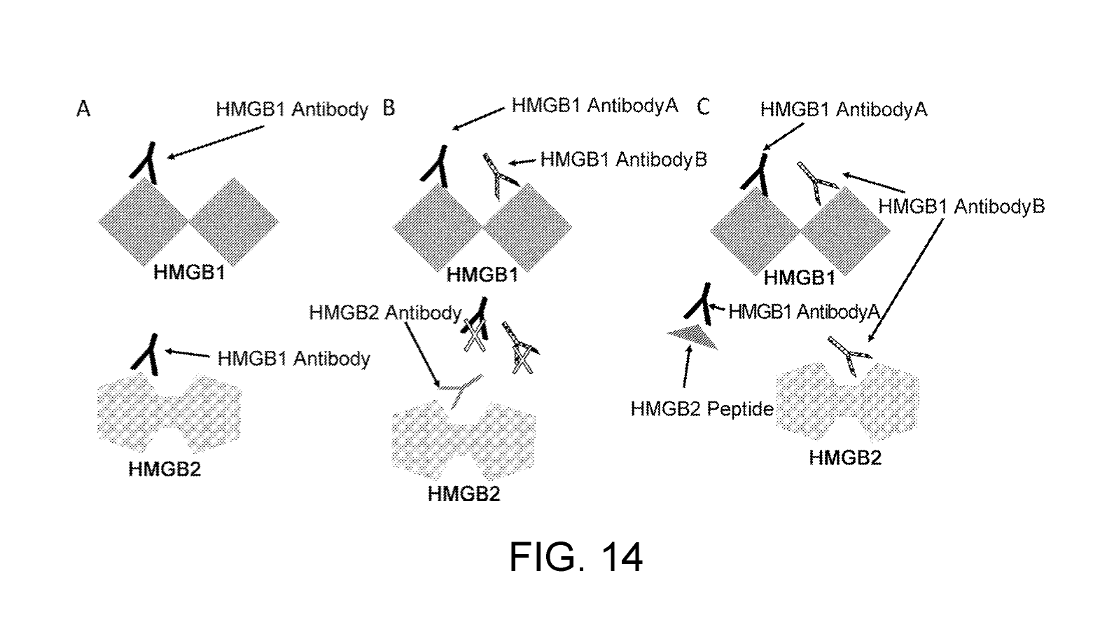

The sequence identity between HMGB1 and HMGB2 in humans is as high as 81.2% at the amino acid level, and many regions share common amino acid sequences (FIG. 13). Therefore, polyclonal antibodies and monoclonal antibodies obtained by immunization with HMGB1 using ordinary methods may bind not only to HMGB1 but also even weakly to HMGB2 having high sequence identity (FIG. 14A). Peptide competition assay (PCA) and a method called blocking peptide are often used as methods for preventing such non-specific binding. When an antigen having the peptide sequence (A) coexists with another antigen having an A′ peptide sequence with a partial modification or substitution of one to several amino acids in A, to specifically recognize A alone, non-specific binding of an A-recognizing antibody to A′ is prevented by making the A′ peptide coexist in the reaction system as a blocking peptide for the A-recognizing antibody, or by allowing A′ to react with an A′-recognizing antibody in advance.

An HMGB2-recognizing HMGB2 antibody reacts with HMGB2 and inhibits reaction of an HMGB1 antibody A with HMGB2 by masking the area around the epitope-like sequence present in HMGB2 which is recognized by the HMGB1 antibody A or by causing steric hinderance. This makes it easy for the HMGB1 antibody to bind to HMGB1 (FIG. 14B). Furthermore, since an antibody molecule has a large molecular weight, steric hinderance takes place readily. Therefore, non-specific reactions by an HMGB1 antibody B and such which recognize different epitopes will be inhibited (FIG. 14B).

On the other hand, an HMGB2 peptide competes with HMGB2, and inhibits binding of the HMGB2-binding HMGB1 antibody A to HMGB2. As a result, the anti-HMGB1 antibody A reacts specifically to HMGB1 (FIG. 14C). However, since the HMGB2 peptide has a small molecular weight, its competitive inhibitory effects may be exhibited to similar epitopes only. That is, it is difficult to inhibit antibodies like the HMGB1 antibody B by peptides (FIG. 14C). In this case, if a peptide of a portion close to the epitope of the antibody is selected, inhibition by the peptide may be possible.

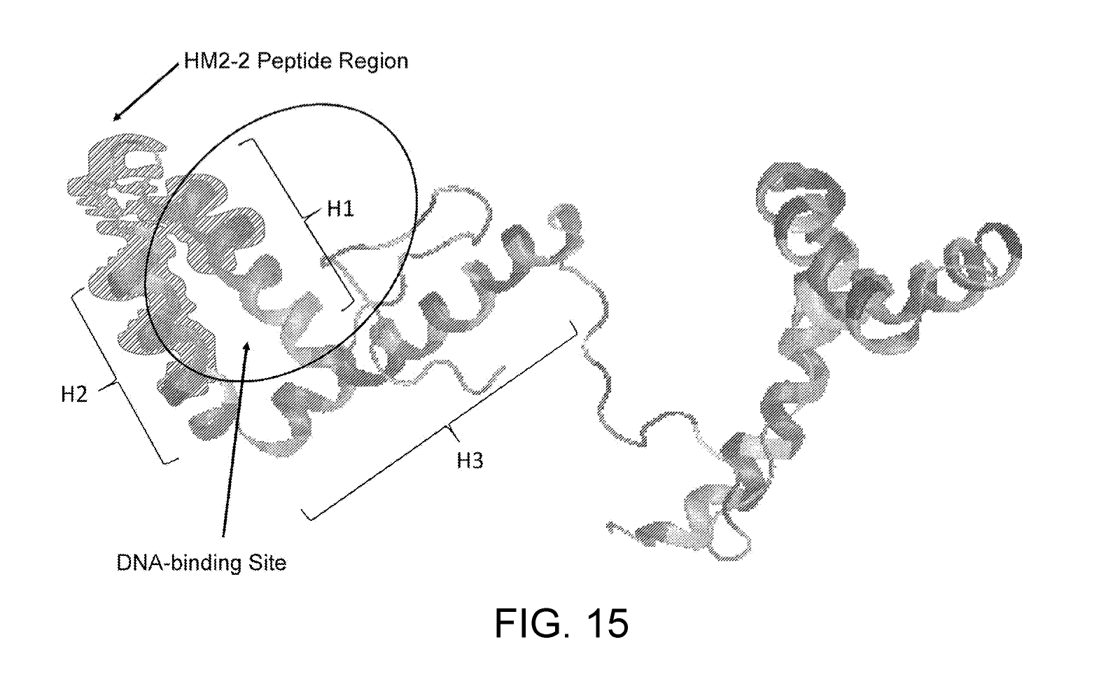

As indicated in the Examples, in the peptide (II) (HM2-2 peptide)-added group, concentration-dependent enhancement of the reaction was observed in HMGB1 measurements. That is, peptide (II) (HM2-2 peptide) of the present invention has effects of enhancing binding between HMGB1 and HMGB1 antibodies, in addition to the activity of inhibiting binding between HMGB2 and the HMGB1 antibodies. This enhancement effect is discussed. The three-dimensional structural model of HMGB1 is shown in FIG. 15. HMGB1 which is a non-histone protein involved in chromatin structure has been known to bind to DNA by surrounding it with the three helical structures (H1 to H3) shown in the figure (DNA binds to the oval region shown in FIG. 15). HM2-2 peptide is a peptide corresponding to the shaded area in the ribbon model of FIG. 15. Furthermore, the HM2-2 peptide region includes a domain structure containing many lysines (K). Such a structure in which basic amino acids such as lysine, arginine (R), and histidine (H) appear successively or every one to two amino acids, is a region readily bound by negatively-charged molecules (HMGB1-binding inhibitory substances) such as heparin (BBXB and such; B refers to a basic amino acid) and phosphatidylserine.

Since DNAs, RNAs, heparin, and such that bind to HMGB1 are macromolecules, they may readily cause steric hindrance towards HMGB1. Therefore, peptides containing the basic amino acids of HMGB1 or HMGB2 can remove steric hinderance against HMGB1 with less dependence on the epitope of the antibody. This should lead to the result that the HM2-2 peptide has enhanced binding of various HMGB1 antibodies.

The use of HMGB2 peptide is necessary to prevent steric hinderance by substances that bind to HMGB1, and also to secure the specificity of reactions by HMGB1 antibodies. This is because, while HMGB1 peptides can remove the binding substances, such peptides will also bind to the antibodies at the same time.

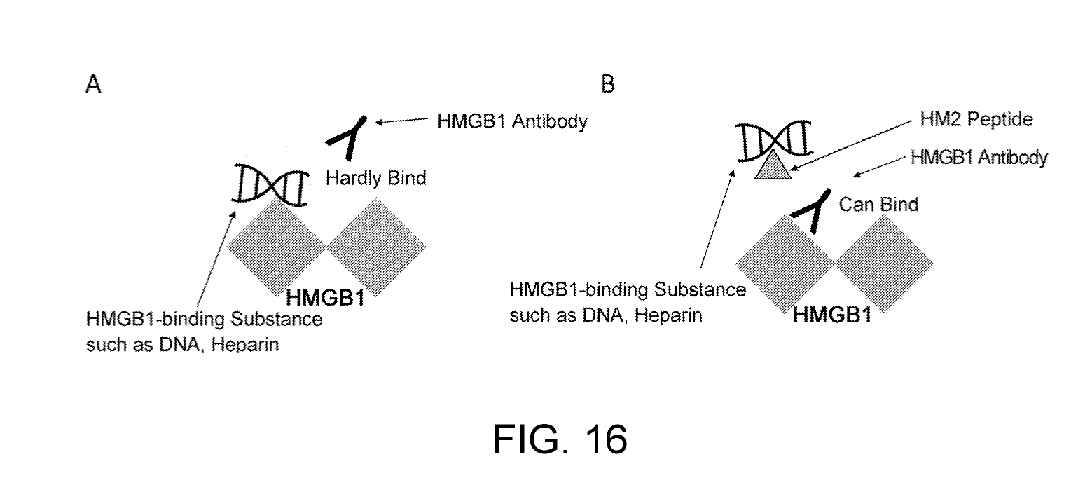

This way, in the present invention, by making the HM2-2 peptide coexist in samples, it binds to minute amounts of HMGB1-binding inhibitory substances such as heparin present in the reaction system. This should remove steric hindrance regarding HMGB1 and make it easy for anti-HMGB1 antibodies to bind to HMGB1 (FIG. 16).

The HMGB2 peptides constituting absorbents of the present invention may have an activity of enhancing binding between HMGB1 and HMGB1 antibodies, in addition to the activity of inhibiting binding between HMGB2 and HMGB1 antibodies. Use of peptides having such activities enables particularly efficient specific measurement or detection of HMGB1.

Accordingly, a peptide having the amino acid sequence of formula (I) or (II), which has been confirmed to be a region having low identity as a result of amino acid sequence comparison between HMGB1 and HMGB2 and also having strong antigenicity according to antigenicity analysis, is a peptide that weakly reacts with HMGB1 antibodies and is expected to suppress binding between HMGB1 antibodies and HMGB2 included in samples. Therefore, such peptides can be used as blocking peptides in immunoassays that use antibodies against HMGB1.

In the present invention, HMGB2 antibodies selectively bind to HMGB2 included in samples, and as a result, inhibit or suppress binding between HMGB2 and HMGB1 antibodies. Therefore, the types and origins of the HMGB2 antibodies used in the present invention are not limited as long as the antibodies have HMGB2-binding properties. For example, antibodies obtained from various immunized animals such as mice, rabbits, and goats can be used. Polyclonal antibodies, anti-serum comprising polyclonal antibodies, monoclonal antibodies, or fragments of these antibodies such as Fab, F(ab′)2, and Fab′, and low-molecular-weight antibodies (for example, scFv, diabody, and sc(Fv)2) can also be used. They can be obtained by methods well known to those skilled in the art.

HMGB2 antibodies of the present invention may be antibodies that have affinity only to HMGB2, or antibodies that have affinity to HMGB1 in addition to HMGB2. However, when using HMGB2 antibodies having affinity also to HMGB1, those having greater binding activity to HMGB2 than to HMGB1 are preferred. Binding activities of the antibodies can be measured by methods well known to those skilled in the art such as enzyme-linked immunosorbent assay (ELISA, EIA), fluoroimmunoassay (FIA), Western blotting, dot blotting, immunoprecipitation methods, radioimmunoassay (RIA), luminescent immunoassay (LIA), enzyme antibody technique, fluorescent antibody technique, immunochromatography method, immunoturbidimetry, latex turbidimetry, and latex agglutination assay.

In the present invention, as long as HMGB2 antibodies can inhibit binding between HMGB2 and HMGB1 antibodies, the sites of the HMGB antigen to which the antibodies bind are not limited. However, since it is preferred that the affinity of HMGB2 antibodies to HMGB2 is greater than the affinity of the HMGB2 antibodies to HMGB1, HMGB2 antibodies used in the present invention are desirably antibodies that recognize the amino acid sequence of formula (I) or (II). The reason is that since these amino acid sequences have low homology to the amino acid sequence of HMGB1 and thus can be expected to produce strong antigenicity of HMGB2.

As shown in the Examples, in the HMGB2 antibody No. 1-added and HMGB2 antibody No. 2-added groups, concentration-dependent enhancement of the reaction was observed in the HMGB1 measurement. More specifically, the HMGB2 antibodies Nos. 1 and 2 of the present invention have an effect of enhancing binding between HMGB1 and HMGB1 antibodies, in addition to an inhibitory activity on binding between HMGB2 and HMGB1 antibodies. The HMGB2 antibodies that constitute the absorbents of the present invention may have an activity of enhancing binding between HMGB1 and HMGB1 antibodies, in addition to an inhibitory activity on binding between HMGB2 and HMGB1 antibodies.

On the other hand, HMGB2-derived peptides of the present invention, which have an activity of inhibiting binding between HMGB2 and HMGB1 antibodies, are preferably peptides comprising amino acid residues that do not match with the corresponding region in HMGB1. This is because these amino acid residues are considered to be important for inhibiting binding between HMGB2 and HMGB1 antibodies. The HMGB2-derived peptides, which comprise the amino acid sequence of formula (I) or (II), of the present invention both comprise amino acids that do not match with the corresponding region in HMGB1.

In the present invention, HMGB2-derived peptides comprising the amino acid sequence of formula (I) or (II) consist of, for example, less than 30 amino acids, preferably less than 25 amino acids, more preferably less than 22 amino acids, and particularly preferably less than 20 amino acids. In the present invention, HMGB2-derived peptides consisting of the amino acid sequence of formula (I) or (II) are particularly preferred, but are not limited thereto.

For example, in the peptide of formula (I) (SEQ ID NO: 3), the 7th amino acid is different from the corresponding region of HMGB1 (while it is lysine/K in HMGB1, it is asparagine/N in HMGB2).

Furthermore, in the peptide of formula (II) (SEQ ID NO: 4), the 12th and 17th amino acids are different from the corresponding regions of HMGB1 (while the 12th amino acid is alanine/A in HMGB1, it is serine/S in HMGB2; and while the 17th amino acid is serine/S in HMGB1, it is alanine/A in HMGB2).

In the HMGB2-derived peptides of the present invention, which have an activity of inhibiting binding between HMGB2 and HMGB1 antibodies and consist of an amino acid sequence having substitution, deletion, addition and/or insertion of one or more amino acids in the amino acid sequence of formula (I) or (II), it is preferred that amino acid residues that do not match with the corresponding region in HMGB1 are conserved, and the other amino acid residues are altered.

Generally, it is known that one or more (for example, three, four, five, or ten) amino acid alterations (for example, conservative substitution, deletion, insertion, and/or addition) in a peptide do not affect the peptide's functions, or may even enhance the functions of the original protein. Amino acids are classified according to their side chain properties into hydrophobic amino acids (A, I, L, M, F, P, W, Y, and V) and hydrophilic amino acids (R, D, N, C, E, Q, G, H, K, S, and T). Furthermore, amino acid side chains can be classified into aliphatic side chains (G, A, V, L, I, and P), hydroxyl group-containing side chains (S, T, and Y), sulfur atom-containing side chains (C and M), carboxylic acid- and amide-containing side chains (D, N, E, and Q), base-containing side chains (R, K, and H), and aromatic group-containing side chains (H, F, Y, and W). Peptides having the amino acid sequence of formula (I) or (II) with alteration of amino acids included therein to other amino acids classified into a group having the same properties are also included in the HMGB2-derived peptides of the present invention, which have an activity of inhibiting binding between HMGB2 and HMGB1 antibodies. However, HMGB2-derived peptides of the present invention, which have an activity of inhibiting binding between HMGB2 and HMGB1 antibodies, may comprise non-conservative alterations as long as effects of suppressing binding between HMGB2 and HMGB1 antibodies are retained.

Methods of obtaining the peptides are not particularly limited. Examples include a method of obtaining a peptide of interest by extraction of HMGB2 by known methods from the body fluid, cells, tissues, organs, or such of humans or other animals. Alternatively, peptide synthesis methods utilizing genetic engineering can also be used, where a DNA fragment encoding the amino acid sequence of interest is incorporated into a vector and the vector is incorporated into Escherichia coli or such to produce the peptide. Furthermore, the above-mentioned peptides can be obtained by peptide synthesis methods, represented by solid-phase peptide synthesis methods, by means of manual procedures or an automatic synthesizer.

When HMGB2-derived peptides having an activity of inhibiting binding between HMGB2 and HMGB1 antibodies are used as the HMGB2 absorbent in the present invention, combinations of HMGB1 antibodies and the peptides are not particularly limited, but for example, the HMGB1 antibodies may be antibodies that recognize as the epitope an amino acid sequence in HMGB1 that corresponds to the amino acid sequence of the HMGB2-derived peptide. Specifically, for example, when a peptide comprising the amino acid sequence of positions 1 to 17 of HMGB2 is used as the HMGB2-derived peptide, the HMGB1 antibody may be an antibody that recognizes as the epitope an amino acid sequence in HMGB1 that corresponds to the amino acid sequence of the HMGB2-derived peptide, namely the amino acid sequence of positions 1 to 17 in HMGB1.

The concentration or amount of the HMGB2 antibody may be preferably 0.5 μg/mL to 500 μg/mL, and particularly preferably 5 μg/mL to 50 μg/mL, but is not limited thereto.

The concentration or amount of the HMGB2-derived peptide may be preferably 10 μg/mL to 1000 μg/mL, and particularly preferably 50 μg/mL to 500 μg/mL, but is not limited thereto.

Furthermore, the HMGB2 absorbents may be either in a liquid form or in a dry form.

In the present invention, the above-mentioned HMGB2 antibodies and the HMGB2-derived peptides may be used as a solution by mixing or diluting with buffers or the like. Solvents used for mixing or dilution may be various aqueous solvents. Examples include purified water, physiological saline solution, or buffers such as Tris buffer, phosphate buffer, or phosphate buffered saline solution, and Good's buffer, but are not limited thereto. Furthermore, the pH of the buffers is not particularly limited, and suitable pH can be appropriately selected. Generally, pH in the range of pH 3 to 12 can be selected and used, but is not limited thereto. Furthermore, for structural protection of the substances to be measured, the solvents may appropriately contain one, two or more kinds of proteins such as bovine serum albumin (BSA), human serum albumin (HSA), and casein; various saccharides; polymers; powdered skimmed milk; various animal sera such as normal rabbit serum; various antiseptics such as sodium azide and antibiotics; and various surfactants such as nonionic surfactants, amphoteric surfactants, and anionic surfactants.

The means for contacting samples with HMGB1 antibodies in the presence of HMGB2 absorbents is also not particularly limited. In the present invention, HMGB2 absorbents, samples, and HMGB1 antibodies can be contacted in any order. Specifically, HMGB2 absorbents and HMGB1 antibodies can be added to a container holding samples (the HMGB2 absorbents and the HMGB1 antibodies can be added in any order; for example, the samples and the HMGB1 antibodies can be contacted in advance and then the HMGB2 absorbents may be contacted with them, or the samples and the HMGB2 absorbents can be contacted in advance and then the HMGB1 antibodies can be contacted with them; or the HMGB2 adsorbents and the HMGB1 antibodies may be mixed with the samples at the same time).

Alternatively, samples and HMGB1 antibodies can be added to a container holding HMGB2 absorbents (the samples and the HMGB1 antibodies can be added in any order; for example, the HMGB2 absorbents and the samples can be contacted in advance and then the HMGB1 antibodies may be contacted with them, or the HMGB2 absorbents and the HMGB1 antibodies can be contacted in advance and then the samples can be contacted with them; alternatively, the samples and the HMGB1 antibodies may be mixed with the HMGB2 adsorbents at the same time).

Furthermore, HMGB2 absorbents and samples can be added to a container holding HMGB1 antibodies (the HMGB2 absorbents and the samples can be added in any order; for example, the HMGB1 antibodies and the HMGB2 absorbents can be contacted in advance and then the samples may be contacted with them, or the HMGB1 antibodies and the samples can be contacted in advance and then the HMGB2 absorbents can be contacted with them; the HMGB2 adsorbents and the samples may be mixed with the HMGB1 antibodies at the same time).

Various other orders are also possible.

In the present invention, the methods of measuring or detecting HMGB1 are not particularly limited, and known techniques can be used. Examples include immunological testing methods such as enzyme-linked immunosorbent assay (ELISA, EIA), fluoroimmunoassay (FIA), Western blotting, dot blotting, immunoprecipitation methods, radioimmunoassay (RIA), luminescent immunoassay (LIA), enzyme antibody technique, fluorescent antibody technique, immunochromatography method, immunoturbidimetry, latex turbidimetry, and latex agglutination assay, but are not limited thereto. Furthermore, measurement or detection in the present invention may be performed manually or by using an apparatus such as an analyzer.

For example, when using enzyme immunoassay, measurement or detection can be performed using a microplate onto which a first HMGB1 antibody is immobilized, HMGB2 absorbents, a second HMGB1 antibody modified with an enzyme such as HRP, washing buffers, and luminescent/chromogenic substrate solutions. Furthermore, HMGB1 can be measured or detected by reacting an enzyme used to modify the second HMGB1 antibody with a substrate of this enzyme under appropriate conditions, and measuring the amount of products of the enzymatic reaction by optical methods.

Alternatively, when using fluoroimmunoassay, measurement or detection can be performed using an optical waveguide or a microplate onto which a first HMGB1 antibody is immobilized, HMGB2 absorbents, a second HMGB1 antibody modified with a fluorescent substance, and washing buffers. Measurement or detection of HMGB1 can be carried out by applying excitation light to the fluorescent substance used to modify the second HMGB1 antibody, and measuring the intensity of the fluorescence emitted by the fluorescent substance. Furthermore, when using radioimmunoassay, HMGB1 can be measured or detected by measuring radiation quantity from a radioactive substance by operations similar to those of the method described above, and when using luminescent immunoassay, HMGB1 can be measured or detected by measuring the amount of luminescence from luminescent reaction systems.

When using western blotting or dot blotting methods, after electrophoresis, transfer membranes or membranes to which samples are directly applied are subjected to blocking with BSA or skimmed milk, and then measurement or detection can be performed using HMGB2 absorbents, an HMGB1 antibody modified with an enzyme such as HRP, washing buffers, and luminescent/chromogenic substrate solutions. A secondary antibody directly labeled with an enzyme, a fluorescent dye, or such may be reacted with a first HMGB1 antibody.

HMGB1 can be measured or detected by reacting the above-mentioned substrate with an enzyme used to modify the HMGB1 antibody or the secondary antibody under appropriate conditions, and measuring the amount of products of the enzymatic reaction by optical methods.

When using immunoprecipitation, HMGB2 absorbents can be added together with blocking agents such as BSA or skimmed milk during reaction of samples with the HMGB1 antibodies and the like. Precipitation is carried out using magnetic beads, agarose substrates, or such directly bound to HMGB1 antibodies, or by using secondary antibodies bound to those beads and such.

Furthermore, when using immunoturbidimetry, a latex turbidimetry, a latex agglutination assay, or the like, HMGB1 can be measured or detected by measuring the transmitted light and the scattered light by an endpoint method or rate method. Also, when immunochromatography is used, the color of labeled substances appearing on the test line can be visually identified. Moreover, instruments such as an analyzer can be used instead of the visual identification.

As the solid-phase carriers to be used in the above immunological assays, solid-phase carriers in the form of beads, microplates, test tubes, sticks, membranes, specimen pieces, or the like made of materials such as polystyrene, polycarbonate, polyvinyl toluene, polypropylene, polyethylene, polyvinyl chloride, nylon, polymethacrylate, polyacrylamide, latex, a liposome, gelatin, agarose, cellulose, sepharose, glass, metal, ceramic, or magnetic material can be used, but are not limited thereto.

As methods for immobilization of HMGB1 antibodies mentioned above on a solid phase, known immobilization methods may be used. For example, methods for physical adsorption include methods of mixing antibodies and carriers in solutions such as buffers to contact them, or methods of contacting carriers with antibodies dissolved in buffers or such, but are not limited thereto.

Furthermore, when immobilizing HMGB1 antibodies by chemical bonding methods, preparations can also be performed according to known methods. Examples of such methods include a method in which the antibodies and carriers are mixed with divalent cross-linking reagents such as glutaraldehyde, carbodiimide, imide ester, or maleimide and contacted with each other to react amino groups, carboxyl groups, thiol groups, aldehyde groups, hydroxyl groups, or the like of both the antibodies and the carriers, but are not limited thereto.

Furthermore, if it is necessary to perform treatments for suppressing non-specific reactions, spontaneous aggregation of the carriers onto which the HMGB1 antibodies are immobilized, and the like, such treatments can be performed according to known methods. Examples of such methods include a method in which the antibody-immobilized surface or inner wall of the carriers is contacted with proteins such as bovine serum albumin (BSA), casein, gelatin, ovalbumin, or salts thereof, surfactants, powdered skimmed milk, or the like to coat the surface or the inner wall of the carriers, but are not limited thereto.

Known modification methods can be used as the method for modifying the above-mentioned second HMGB1 antibody with labeling substances. Examples of physical adsorption methods include, but are not limited to, a method in which the second HMGB1 antibody and labeling substances are mixed and contacted with each other in solutions such as buffers and a method in which the antibody dissolved in buffers and the like is contacted with labeling substances. For example, when the labeling substance is gold colloid or latex, physical adsorption methods are effective. Antibodies labeled with gold colloid can be obtained by mixing the antibodies and gold colloid in buffers to contact them with each other.

Furthermore, when modifying the second HMGB1 antibody with labeling substances by chemical bonding methods, the preparation can be carried out according to known methods. Examples of such methods include, but are not limited to, a method in which the antibody and labeling substances are mixed with divalent cross-linking reagents such as glutaraldehyde, carbodiimide, imide ester, or maleimide and contacted with each other to react amino groups, carboxyl groups, thiol groups, aldehyde groups, hydroxyl groups, or the like of both the antibody and the labeling substances. For example, when the labeling substance is a fluorescent substance, an enzyme, or a chemiluminescent substance, a chemical bonding method is effective.

Furthermore, if it is necessary to perform treatments for suppressing non-specific reactions, spontaneous aggregation of the antibody modified with labeling substances, and the like, such treatments can be performed according to known methods. Examples of such methods include, but are not limited to, a method in which the antibodies conjugated with labeling substances are contacted with proteins such as bovine serum albumin (BSA), casein, gelatin, ovalbumin, or salts thereof, surfactants, powdered skimmed milk, or the like to coat the antibodies.

For labeling substances, when an enzyme immunoassay is carried out, without being limited to the following, peroxidase (POD), alkaline phosphatase (ALP), β-galactosidase, urease, catalase, glucose oxidase, lactate dehydrogenase, amylase, or the like can be used.

When a fluorescent immunoassay is carried out, without being limited to the following, fluorescein isothiocyanate, tetramethylrhodamine isothiocyanate, substituted rhodamine isothiocyanate, dichlorotriazine isothiocyanate, cyanine, merocyanine, or the like can be used.

When a radioimmunoassay is carried out, without being limited to the following, tritium, iodine-125, iodine-131, or the like can be used.

When a luminescence immunoassay is carried out, without being limited to the following, luminol compounds, luciferase compounds, acridinium esters, dioxetane compounds, or the like can be used.

When immunochromatography, immunoturbidimetry, latex turbidimetry, and latex agglutination assay are carried out, without being limited to the following, particles can be used, which are made of materials such as polystyrene, styrene-styrene sulfonate copolymer, acrylonitrile-butadiene-styrene copolymer, vinyl chloride-acrylic acid ester copolymer, vinyl acetate-acrylic acid copolymer, polyacrolein, styrene-methacrylic acid copolymer, styrene-glycidyl (meth)acrylic acid copolymer, styrene-butadiene acid copolymer, methacrylic acid polymer, acrylic acid polymer, latex, gelatin, liposome, microcapsule, silica, alumina, carbon black, metal compound, metal, metal colloid, ceramic, or magnetic material.

Furthermore, the present invention relates to methods of diagnosing sepsis or sepsis-related diseases such as systemic inflammation, which comprise the step of measuring or detecting HMGB1 in samples using methods of measuring or detecting HMGB1 in samples, comprising the step of contacting the samples with HMGB1 antibodies in the presence of HMGB2 absorbents. In the diagnosis methods of the present invention, when HMGB1 is detected, the subject is determined to be affected with sepsis or sepsis-related diseases. In the present invention, the methods may include a step of administering known therapeutic agents for sepsis or related diseases, or carrying out known therapies on a subject in which HMGB1 has been detected.

While further research is necessary regarding the correlation between blood concentration of HMGB1 and the severity of sepsis and sepsis-related diseases, blood concentration of HMGB1 is known to be high in sepsis patients (Wang 1999/Non-patent Document 1). Furthermore, the blood HMGB1 concentration has been reported to show high correlation with the SOFA score which is one of the diagnostic criteria for sepsis, the lactate level, the procalcitonin level, and such (Gibot 2007/Non-patent Document 4). Furthermore, it has been reported that after onset of sepsis, the blood HMGB1 concentration decreased with time in patients who did not die, but the blood HMGB1 concentration increased with time in patients with poor prognosis and died after onset of the disease (Gibot 2007/Non-patent Document 4). In addition, there has been a report of a case of therapy where blood purification therapy performed on sepsis patients resulted in recovery of elevated HMGB1 concentrations to normal levels and discharge of the patients from the hospital (Nakamura 2011/Non-patent Document 5). This way, blood HMGB1 concentration serves as important information for initiation of and withdrawal from various treatments.

Furthermore, the present invention relates to kits for measuring or detecting HMGB1 in samples, which comprises at least a first reagent comprising an HMGB1 antibody and a second reagent comprising an HMGB2 absorbent. The present invention also relates to kits for diagnosis of sepsis, which comprises at least a first reagent comprising an HMGB1 antibody and a second reagent comprising an HMGB2 absorbent. These kits of the present invention are characterized in that they are used for contacting a sample with an HMGB1 antibody in the presence of an HMGB2 absorbent. The kits of the present invention are not particularly limited so long as they are composed such that binding between HMGB1 in a sample and an HMGB1 antibody takes place in the presence of an HMGB2 absorbent.

As described above, the means for contacting samples with HMGB1 antibodies in the presence of HMGB2 absorbents are not particularly limited. For example, HMGB2 absorbents, samples, and HMGB1 antibody-immobilized carriers may be added in any order, and the HMGB2 absorbents can be added in a liquid form or in a dry form. That is, to a container holding any one or two of the samples, the HMGB2 absorbents, and the HMGB1 antibody-immobilized carriers, the other two or one may be added. In this case, the other two may be added in any order, and they may be added at the same time. Furthermore, the HMGB2 absorbents, the samples, and the HMGB1 antibody-immobilized carriers can be contacted at the same time. Various other means can be used as well.

Furthermore, for the constitution of the kits of the present invention, HMGB2 absorbents may be combined with other reagents or they can be adhered to solid phases in advance. For example, when combined in advance with other reagents, the reagents to be combined include buffers necessary for binding between HMGB1 in the samples and the HMGB1 antibodies, sample dilution solutions, solutions of HMGB1 antibodies containing labeling substances such as an enzyme, reagents containing substances that generate a signal such as color development, reagents containing substances involved in generation of a signal such as color development, reagents containing substances for calibration, and reagents containing substances used for accuracy control. Examples of the solid phase include carriers and test papers to be used in the immunological testing kits, microplates, glass plates, microtubes, filter papers, and polymer resins.

In the present invention, kits refer to kits containing reagents or such necessary for measuring or detecting HMGB1. Kits of the present invention also include sets of various reagents necessary for measuring or detecting HMGB1, disposable kits including packs of various reagents necessary for measuring or detecting HMGB1, microplate-type testing kits for measuring multiple samples simultaneously, and immunochromatography and test papers, which contain integrated reagents and such and allow determination of the results by visual observation.

An example of the form of disposable kits can be that constituted of testing containers packed with the first HMGB1 antibody-immobilized spherical- or rod-shaped carriers, HMGB2 adsorbents, reagent dilution solutions, a second HMGB1 antibody modified with an enzyme such as ALP, washing solutions, luminescent substrate solutions, and such, but it is not limited thereto.

The form of the testing containers is not particularly limited as long as HMGB1 in the samples can be measured or detected. Examples include, but are not limited to, boat-shaped containers in which multiple reaction chambers and reagent storage chambers are aligned, and flow-channel-type containers in which grooves are provided on a sheet-type substrate and reaction chambers and storage chambers are connected by flow channels. Furthermore, while the size of the testing container is not particularly limited, but for use by incorporation into an automated analyzer and such, a small size such as approximately 10 cm×10 cm or smaller is desired. Furthermore, to avoid intrusion of foreign matter into the reaction chambers and evaporation/deterioration of reagents packed in the reagent storage chambers, the upper portion of each chamber may be sealed. Examples include a method of adhesion of aluminum foil, polymeric film, or such to the upper portion of the reaction chambers and storage chambers of the testing container. In particular, sealing with aluminum foil is preferred since it can be easily opened using the tip of a dispenser chip or the punching structure of an analyzer. The material for the testing container is not particularly limited as long as it does not inhibit the reaction for measuring the substances to be measured. Examples include, but are not limited to, polystyrene resins, polyethylene resins, and polypropylene resins.

Furthermore, the following constitution can be considered as a form of the microplate-type kits. For example, the constitution involves the first HMGB1 antibody-immobilized microplates accompanied by HMGB2 adsorbents, sample dilution solutions, a second HMGB1 antibody modified with an enzyme such as HRP, washing solutions, chromogenic substrate solutions and such in their respective reagent bottles. Alternatively, HMGB2 adsorbents may be included in advance in sample dilution solutions or may be allowed to adhere to microplates in a dry state.

A suitable form of immunochromatography is lateral flow composed of housing cases, sample pads, conjugation pads, membrane filters, and absorption pads. Lateral flow is prepared by the following procedure. First, a first HMGB1 antibody labeled with gold colloid or colored beads is prepared, applied to the conjugation pad, and dried. On the other hand, a second HMGB1 antibody is applied to the test line of the membrane filter and dried. Furthermore, a third antibody that specifically recognizes the second HMGB1 antibody is applied to the control line of the membrane filter and dried. Finally, to the resulting filter, the above-mentioned conjugation pad, sample pad, and absorption pad are attached to form the lateral flow. Furthermore, the above-mentioned lateral flow may be accompanied by HMGB2 absorbents to make up an immunochromatographic test kit. Alternatively, HMGB2 absorbents can be applied to the sample pad or conjugation pad and dried, and the resulting pad can be attached to the above-mentioned membrane filter, to thereby make up an immunochromatographic test kit.

A more specific embodiment of the present invention is, for example, a kit for measuring or detecting HMGB1 which comprises an HMGB2 absorbent-containing solution and an HMGB1 antibody-immobilized carrier so that, at the time of the binding reaction between the HMGB1 antibody and HMGB1 in the samples, the concentration of the above-mentioned absorbent can be made to be 5 μg/mL to 50 μg/mL where the absorbent is a HMGB2 antibody and 50 μg/mL to 500 μg/mL where the absorbent is a peptide of formula (I) or (II). Such a kit can be used for any immunological test such as enzyme-linked immunosorbent assay (ELISA, EIA), fluoroimmunoassay (FIA), radioimmunoassay (RIA), luminescent immunoassay (LIA), enzyme antibody technique, fluorescent antibody technique, immunochromatography method, immunoturbidimetry, latex turbidimetry, and latex agglutination assay. Furthermore, depending on the respective methods, HMGB1 antibodies can be modified appropriately or may be fixed onto carriers. Furthermore, when a kit of the present invention is used for latex-beads, microtubes, or immunochromatography methods, it may contain housing cases, sample pads, conjugation pads, membrane filters, absorption pads, colloids, and such.

Alternatively, another example includes kits for measuring or detecting HMGB1, which comprise HMGB2 absorbent-attached and HMGB1 antibody-immobilized microplates so that, at the time of the binding reaction between the HMGB1 antibody and HMGB1 in samples, the concentration of the above-mentioned absorbent can be made to be 5 μg/mL to 50 μg/mL where the absorbent is an HMGB2 antibody and 50 μg/mL to 500 μg/mL where the absorbent is a peptide of formula (I) or (II). Such a kit can be used for immunological tests such as enzyme-linked immunosorbent assay (ELISA, EIA), fluoroimmunoassay (FIA), radioimmunoassay (RIA), and luminescent immunoassay (LIA). Furthermore, depending on the respective methods, the HMGB1 antibodies may be modified appropriately. Additionally, immunological test kits may contain substrate solutions or such.

The present invention also relates to reagents for measuring or detecting HMGB1 in samples, which comprise at least an HMGB1 antibody and an HMGB2 absorbent. Furthermore, the present invention relates to diagnostic reagents for sepsis, which comprise at least an HMGB1 antibody and an HMGB2 absorbent. These reagents of the present invention are characterized in that they are used for contacting samples with the HMGB1 antibody in the presence of the HMGB2 absorbent.

In the present invention, the form of the reagents is not particularly limited and may have a volume and form that match the test of interest, and it may be a solution, which contains the HMGB2 antibody or the peptide or both, or may be in the form of a tablet or powder thereof, or in a dry form attached to a container.

Furthermore, the present invention relates to peptides comprising the amino acid sequence of formula (I) or (II), peptides consisting of the amino acid sequence of formula (I) or (II), or peptides having an activity of inhibiting binding between HMGB2 and an HMGB1 antibody, which consist of an amino acid sequence having substitution, deletion, addition and/or insertion of one or more (for example, two, three, four, five, or ten) amino acids in the amino acid sequence of formula (I) or (II).

| | | |
|---|---|---|
| (I): | MGKGDPNKPRGKMSSYA | (SEQ ID NO: 3) |
| (II): | CREEHKKKHPDSSVNFAEFS | (SEQ ID NO: 4) |

These peptides can be used to detect HMGB1. Specifically, by contacting samples containing these peptides with HMGB1 antibodies (for example, an HMGB1 antibody having affinity to both HMGB2 and HMGB1), HMGB1 alone can be detected specifically.

Furthermore, the present invention relates to samples which contain

a peptide comprising the amino acid sequence of formula (I) or (II) mentioned above,
a peptide consisting of the amino acid sequence of formula (I) or (II), or
a peptide having an activity of inhibiting binding between HMGB2 and an HMGB1 antibody, which consists of an amino acid sequence having substitution, deletion, addition and/or insertion of one or more (for example, two, three, four, five, or ten) amino acids in the amino acid sequence of formula (I) or (II). Such samples can also be used to specifically detect HMGB1 alone using HMGB1 antibodies (for example, an HMGB1 antibody having affinity to both HMGB2 and HMGB1).

Furthermore, the present invention relates to use of HMGB1 antibodies and HMGB2 absorbents in producing agents for measuring or detecting HMGB1. The present invention also relates to use of HMGB1 antibodies and HMGB2 absorbents in producing agents for measuring or detecting HMGB1, which comprises the step of contacting samples with HMGB1 antibodies in the presence of HMGB2 absorbents.

Furthermore, the present invention relates to HMGB1 antibodies and HMGB2 absorbents for use in measuring or detecting HMGB1 in samples. The present invention also relates to HMGB1 antibodies and HMGB2 absorbents for use in measuring or detecting HMGB1 in samples, which comprise contacting samples with HMGB1 antibodies in the presence of HMGB2 absorbents.

All prior art references cited herein are incorporated by reference into this description.

### EXAMPLES

Herein below, the present invention will be described in detail with reference to the Examples, but it is not to be construed as being limited thereto.

### [Example 1] HMGB1-Specific Measurement Method by Competitive ELISA Using an Anti-HMGB2 Antibody

Competitive ELISA was performed in the presence of an anti-HMGB2 antibody to specifically measure HMGB1, and whether nonspecific reaction to HMGB2 can be decreased was examined.

A 100 μL portion of 10 μg/mL anti-HMGB1 antibody HMa166 was added to a 96-well ELISA plate (Maxsorp, Nunc). Then, immobilization was carried out according to a standard method, and blocking was performed by dispensing 200 μL of ImmunoBlock (DS Pharma Biomedical) diluted five-fold in purified water. After washing with TBSt (10 mM Tris pH 7.4, 150 mM NaCl, 0.05% Tween-20), HMGB2 was added at 1,400 ng/mL, the mixture was allowed to react at room temperature for two hours followed by washing with TBSt, and an HRP-labeled anti-HMGB1 antibody CP11-1 was added. Then, after washing, chromogenic substrate TMBZ (KPL) was added according to a standard method. The reaction was stopped using phosphoric acid, and the absorbance at 450 nm was measured using a microplate reader. Furthermore, as a competitive agent, commercially available anti-HMGB2 antibodies shown in Table 1 were added to the reaction system at 10 μg/mL, and impairment of the reaction due to anti-HMGB2 antibodies was observed.

TABLE 1
Anti-HMGB2 Antibodies used for Competitive ELISA

| | | | Manufac- | Commodity |
|---|---|---|---|---|
| No. | Product Name | Antigen | turer | Code |
| 1 | Anti-HMGB2, | Human HMGB2 | Abnova | H00003148-M03 |
| | Mono (3C7) | | | |
| 2 | Anti-HMGB2, | Human HMGB2 | Abnova | H00003148-M06 |
| | Mono (3F2) | | | |
| 3 | Anti-HMGB2, | Human HMGB2 | Abnova | H00003148-M07 |
| | Mono (X1) | | | |

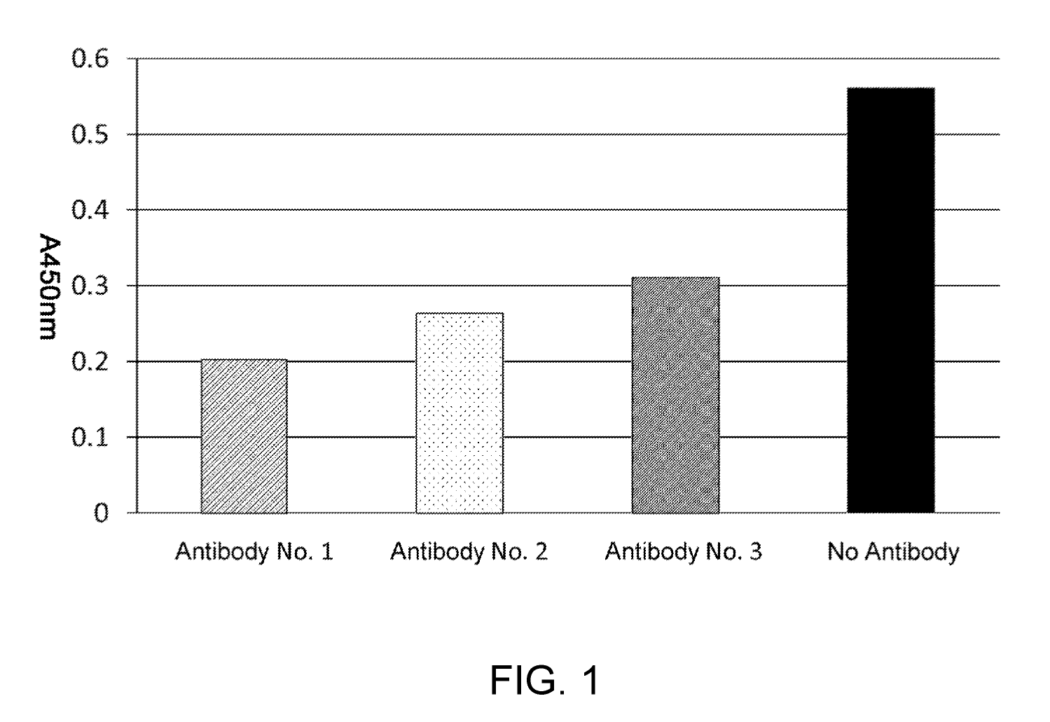

The results of the measurement of HMGB2 by sandwich ELISA using MHa166 antibody and CP11-1 antibody, which were obtained by immunization with HMGB1, showed that the reaction was not specific to HMGB1 and nonspecific reaction to HMGB2 took place as well. At 1,400 ng/mL HMGB2, the absorbance was 0.56 (FIG. 1; no absorbent). However, when 10 ng/mL anti-HMGB2 antibody was allowed to coexist in this reaction system, while absorbance differed depending on the antibodies used, the respective absorbances decreased to approximately 50% compared to the absorbance in the absence of an absorbent. Coexisting anti-HMGB2 antibody was found to have effects of suppressing the nonspecific reaction.

To investigate what kind of impacts on HMGB1 measurements are brought by the commercially available anti-HMGB2 antibodies, which showed effects of absorbing the non-specific reaction to HMGB2, calibration curves were produced using 0 to 50 μg/mL HMGB1 under conditions where the anti-HMGB2 antibodies shown in Table 1 were added at 10 μg/mL to the ELISA system in which anti-HMGB1 antibody MHa166 was immobilized, blocking with ImmunoBlock (DS Pharma Biomedical) diluted five-fold in purified water was performed, and HRP-labeled anti-HMGB1 antibody CP11-1 was used. Then, the effects of each anti-HMGB2 antibody were observed.

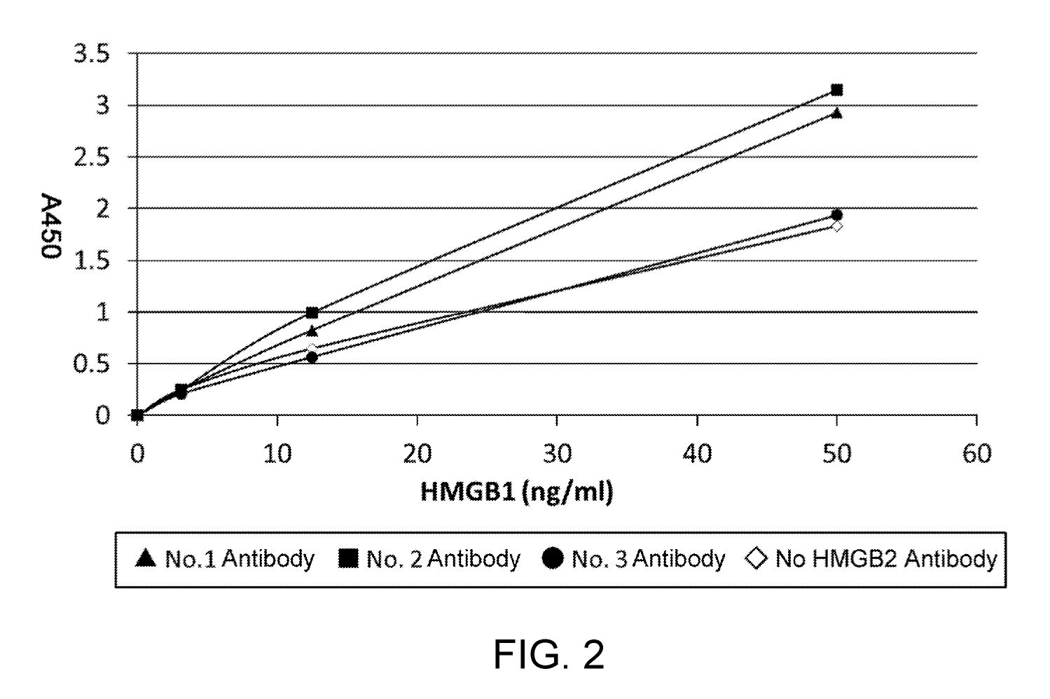

When considering the amino acid sequence homology between HMGB1 and HMGB2, the reaction was predicted to be apparently impaired since some of the added anti-HMGB2 antibodies bind to HMGB1 and block binding of an HMGB1 antibody due to steric hinderance and such; however, the result obtained by adding an anti-HMGB2 antibody was that HMGB1 measurements were not affected at all when the No. 3 HMGB2 antibody was used (FIG. 2). Furthermore, when anti-HMGB2 antibody No. 1 or No. 2 was added, the measured values of HMGB1 even slightly increased (FIG. 2). Although the details of this amplified effect are unclear, at least anti-HMGB2 antibody No. 3 was found not to affect HMGB1 measurements.

Furthermore, 0 to 1,300 μg/mL HMGB2 was measured by sandwich ELISA using the MHa166 antibody and the CP11-1 antibody, which were obtained by immunization with HMGB1, to observe how much HMGB2 reacts in ELISA using the MHa166 antibody and the CP11-1 antibody. Furthermore, with respect to the HMGB2 calibration curve prepared in this manner, whether absorption effects can be obtained by addition of 10 μg/mL anti-HMGB2 antibody was observed.

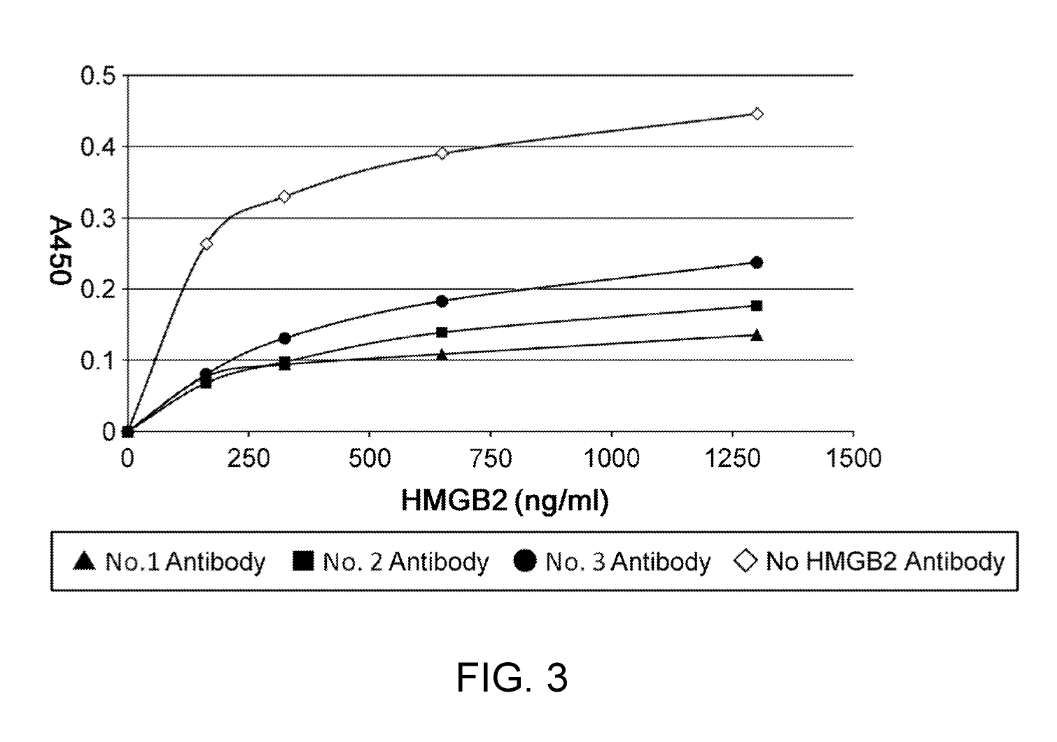

As a result, reaction to HMGB2 was also observed in sandwich ELISA using the MHa166 antibody and the CP11-1 antibody, which were obtained by immunization with HMGB1, and this reaction resulted in an absorbance value (A450) of as high as approximately 0.26 for 162.5 ng/mL HMGB2 (FIG. 3). On the other hand, when various anti-HMGB2 antibodies were allowed to coexist in this system, while absorbance differed depending on the antibodies, the absorbance value (A450) decreased to approximately 0.08 for 162.5 ng/mL HMGB2. The antibody that did not affect HMGB1 measurements and showed high absorption effects was the anti-HMGB2 antibody No. 3 (FIGS. 2 and 3).

Depending on actual measurement samples derived from living organisms, HMGB1 and HMGB2 are assumed to be mixedly present, and in that case, whether HMGB1 alone can be specifically measured was observed by allowing an anti-HMGB2 antibody to absorb the mixed HMGB2. Pseudo-mixed samples were prepared by mixing HMGB1 subjected to two-fold serial dilutions from 50 ng/mL with HMGB2 subjected to two-fold serial dilutions from 1820 ng/mL according to Table 2.

TABLE 2
Pseudo-prepared Mixed Samples of HMGB1 and HMGB2

| HMGB1 (ng/mL) | HMGB2 (ng/mL) | HMGB1/2 (ng/mL) |
|---|---|---|
| 50 | 1820 | 1870 |
| 25 | 910 | 935 |
| 12.5 | 455 | 467.5 |
| 6.3 | 227.5 | 233.8 |
| 3.1 | 113.8 | 116.9 |
| 1.6 | 56.9 | 58.4 |
| 0.8 | 28.4 | 29.2 |
| 0 | 0 | 0 |

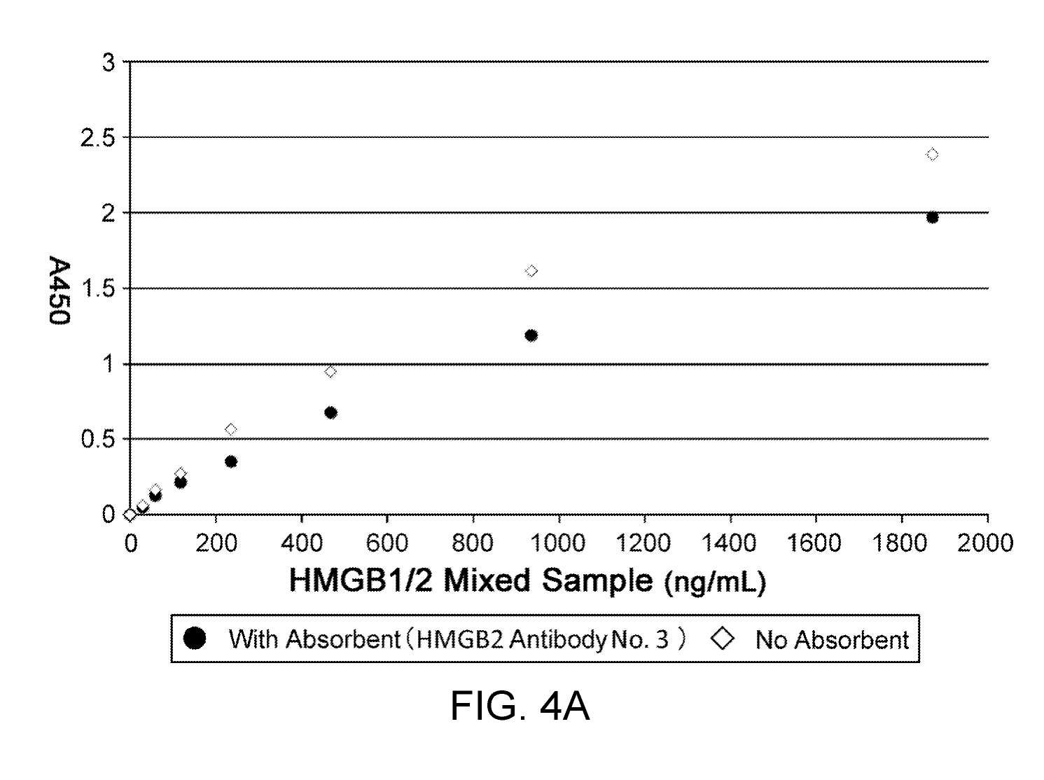

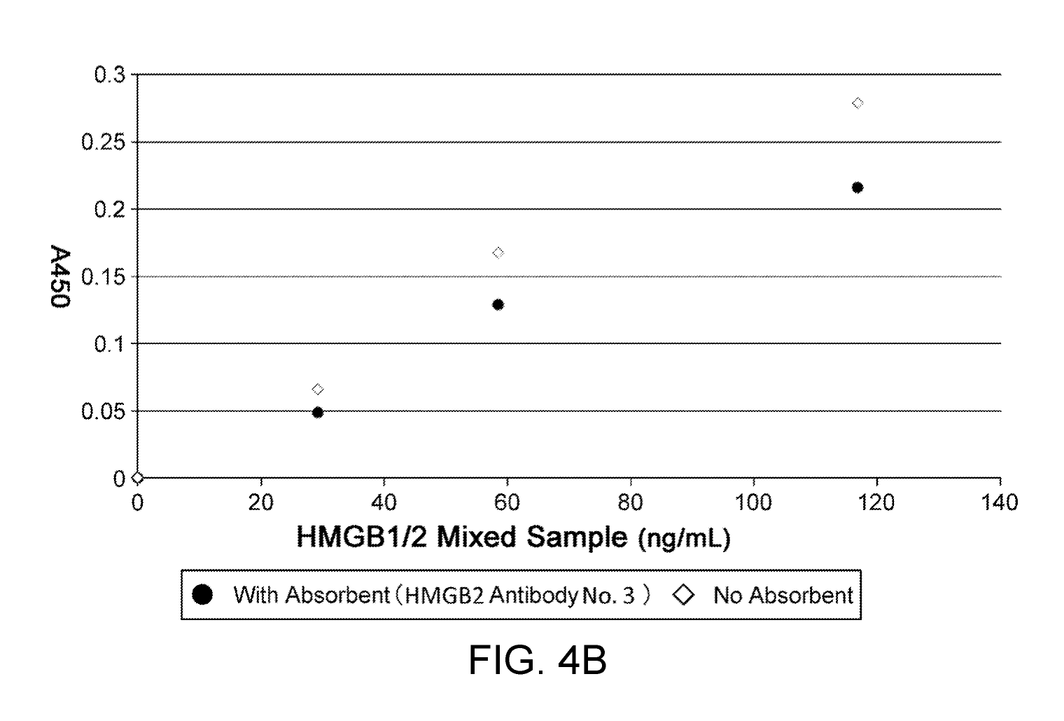

Mixed samples were measured by sandwich ELISA using the MHa166 antibody and the CP11-1 antibody in the presence or absence of No. 3 anti-HMGB2 antibody. The results are shown in FIG. 4A. Furthermore, the experiment results obtained in the 120 ng/mL or lower range for the HMGB1-HMGB2 mixture used in this experiment are shown in detail in FIG. 4B. This way, it was found that in the absence of the absorbent, measured values were apparently larger than the actual HMGB1 concentration. On the other hand, in the group to which No. 3 anti-HMGB2 antibody was added as an absorbent at 10 μg/mL, it was found that nonspecific reaction to HMGB2 can be suppressed and HMGB1 alone can be measured specifically.

Furthermore, a similar experiment was performed to measure, in addition to the HMGB1 and HMGB2 mixture (HMGB1/2), samples prepared by adding an anti-HMGB2 antibody, which is an absorbent, to HMGB1, at the same concentration as that of HMGB1 in the mixture, and to examine how much deviation from the actual concentration was observed.

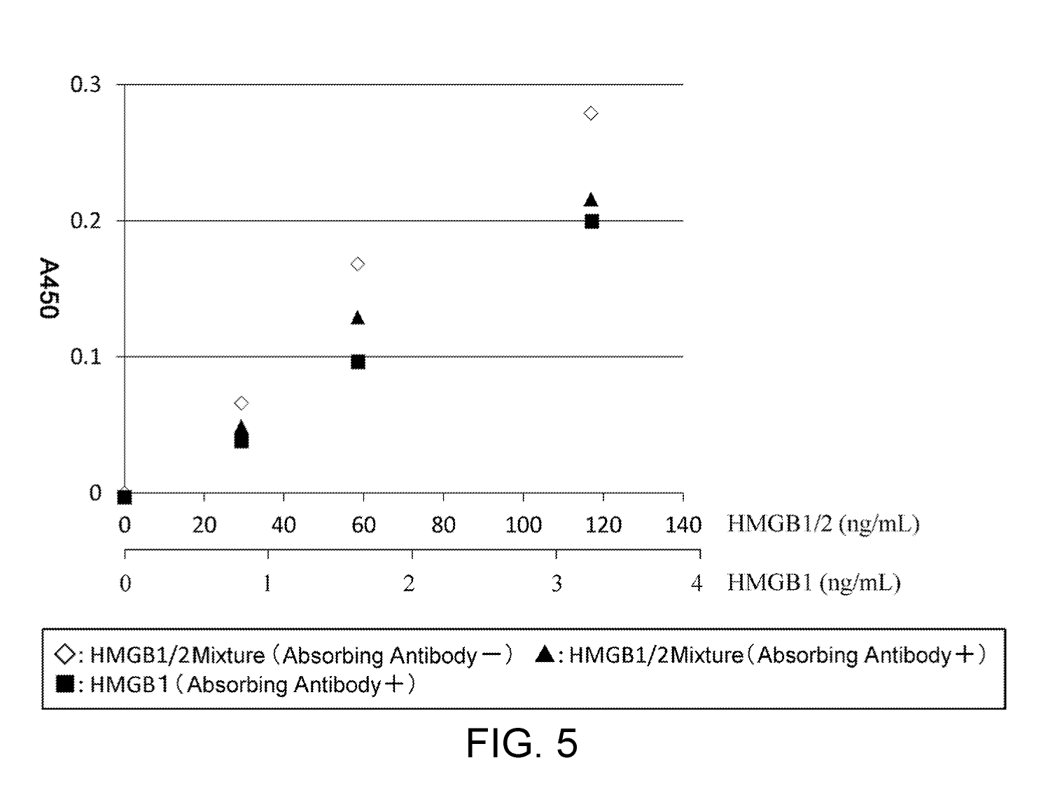

As shown in FIG. 5, similarly to the experimental results of FIG. 4, apparently high values were shown for the HMGB1 and HMGB2 mixture, and when an anti-HMGB2 antibody was added thereto as the absorbent, nonspecific reaction towards HMGB2 was suppressed. In the latter results, the measured values almost matched or were close to the values obtained by measuring HMGB1 alone, and substantially matched the calibration curve produced using HMGB1. Accordingly, addition of an anti-HMGB2 antibody was found to enable HMGB1-specific measurements.

Furthermore, whether commercially available anti-HMGB2 antibodies can suppress nonspecific reactions to HMGB2 even when the labelled antibody is changed was examined. Anti-HMGB1 antibody HMa166 at 10 μg/mL was immobilized onto a 96-well ELISA plate (Maxsorp, Nunc), and blocking was performed by dispensing 200 μL of ImmunoBlock (DS Pharma Biomedical) diluted five-fold in purified water. After washing with TBSt (10 mM Tris pH 7.4, 150 mM NaCl, 0.05% Tween-20), HMGB1 was added at 50 ng/mL or HMGB2 was added at 1,820 ng/mL, and additionally commercially available anti-HMGB2 antibody No. 3 was added at a final concentration of 10 μg/mL as a competing agent to the reaction system. The mixture was allowed to react at 37° C. for two hours followed by washing with TBSt. After adding HMGB1 polyclonal antibody No. 12 or No. 14 obtained by immunizing rabbits with a synthetic human HMGB1 peptide or a recombinant human HMGB1, each of which had been labeled with HRP, the mixture was reacted at room temperature for one hour. After the reaction, the plate was washed with TBSt, and chromogenic substrate TMBZ (KPL) was added according to a standard method, the reaction was stopped with phosphoric acid, and the absorbances at 450 nm were measured using a microplate reader, to observe impairment of the reaction due to the anti-HMGB2 antibody.

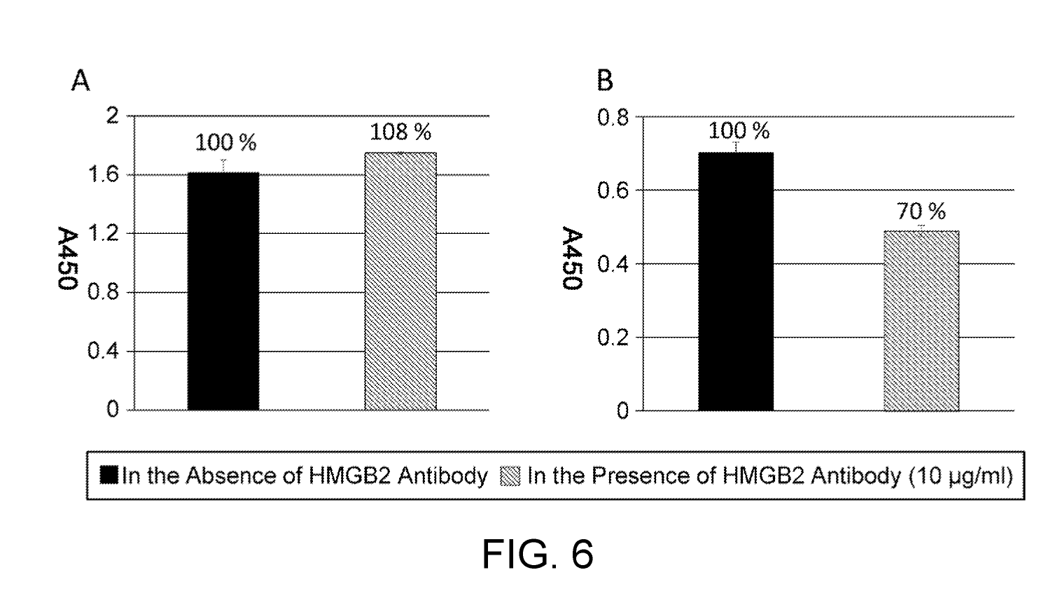

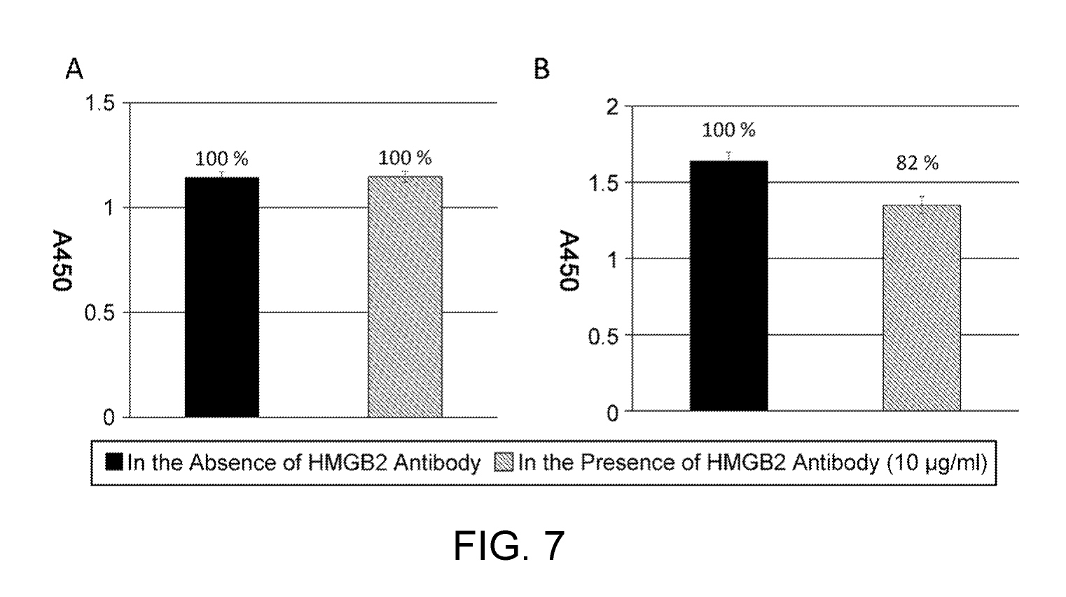

As a result, even when either HMGB1 polyclonal antibody No. 12 or No. 14 was used as the labeled antibodies, the anti-HMGB2 antibody showed no significant effect on HMGB1 measurements (FIGS. 6A and 7A). On the other hand, when using a high concentration of HMGB2, nonspecific reaction to HMGB2 was found in either case of using polyclonal antibody No. 12 or No. 14; however, when an anti-HMGB2 antibody was allowed to coexist, the reaction was impaired to 70% for No. 12 polyclonal antibody, and to 82% for No. 14 polyclonal antibody, indicating that absorption effects by the anti-HMGB2 antibody was obtained in the respective cases (FIGS. 6B and 7B).

### [Example 2] HMGB1-Specific Measurement Method by Competitive ELISA Using HMGB2 Peptides

In the case of experiments using anti-phosphorylation state-specific antibodies and such, for preventing nonspecific reactions, there are methods in which non-phosphorylated peptides are pre-incubated with the antibodies in advance or are allowed to coexist with the antibodies, and such methods are called Peptide Competition Assays (PCA: peptide competition methods) (Non-patent Document 3). Expecting similar effects, whether specific measurement of HMGB1 by addition of HMGB2 peptides is possible was examined.

From regions in the amino acid sequence of HMGB2 which comprise amino acid sequences different from those of HMGB1 and have strong antigenicity according to an antigenicity search, six types of peptides were designed, and among them, two types of peptides were used in the experiments:

| | | |
|---|---|---|
| (I): | MGKGDPNKPRGKMSSYA | (SEQ ID NO: 3) (HM2-1) |
| (II): | CREEHKKKHPDSSVNFAEFS | (SEQ ID NO: 4) (HM2-2) |

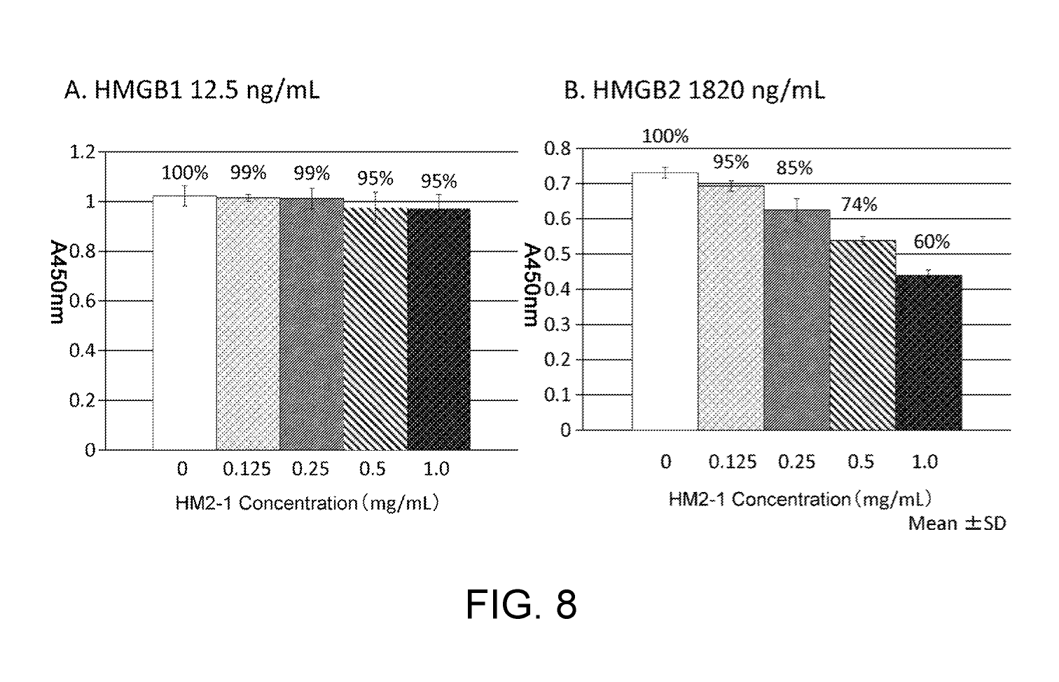

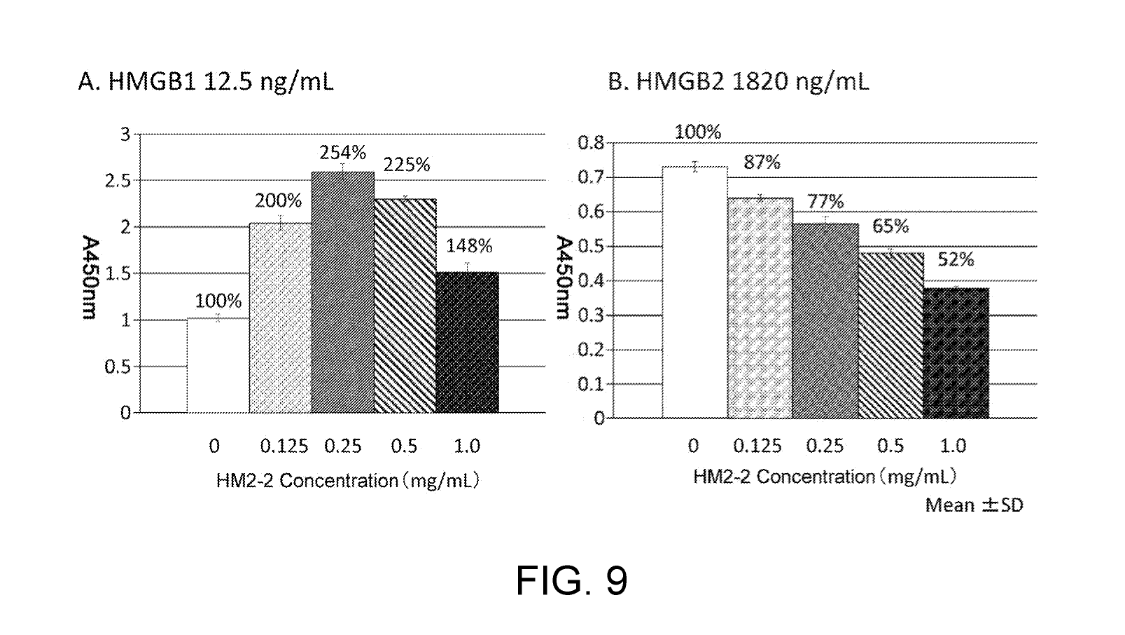

By means of an ELISA system using immobilized anti-HMGB1 antibody MHa166 and an HRP-labeled anti-HMGB1 antibody CP11-1, it was examined whether nonspecific reactions by HMGB2 will be decreased when the reaction is achieved by 1,820 ng/mL HMGB2 in the presence of the HMGB2 peptide at 0 to 1.0 mg/mL (FIGS. 8B and 9B). In addition, effects of the coexisting HMGB2 peptide on HMGB1 measurements were similarly examined using 12.5 ng/mL HMGB1 (FIGS. 8A and 9A).

As a result, the HMGB2 peptide HM2-1 hardly affected the measured values of HMGB1, and was able to absorb nonspecific reaction to HMGB2 in a concentration-dependent manner. This revealed that use of the peptide can absorb nonspecific reactions and enables HMGB1-specific measurements (FIG. 8).

Furthermore, HMGB2 peptide HM2-2 not only absorbed nonspecific reactions to HMGB2 in a concentration-dependent manner (FIG. 9B), but also showed concentration-dependent reaction enhancement in HMGB1 measurements (FIG. 9A).

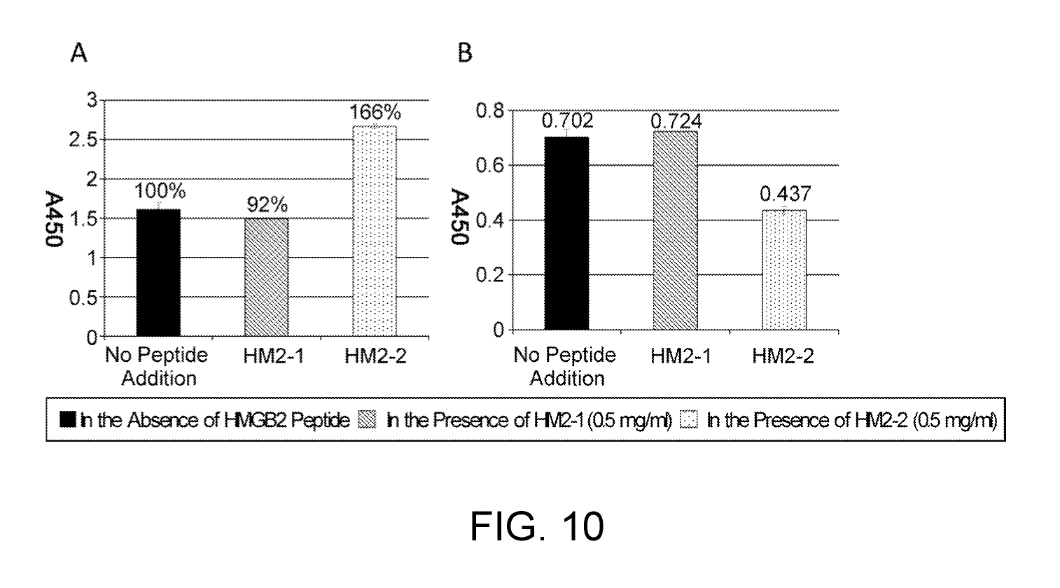

Next, whether similar effects can be obtained with antibodies other than CP11-1 was examined. It was examined how the reactions achieved by 50 ng/mL HMGB1 change when HMGB1 polyclonal antibodies No. 12 and No. 14, which were obtained by immunizing rabbits with a synthetic human HMGB1 peptide or a recombinant human HMGB1, were HRP-labeled and an HMGB2 peptide was allowed to coexist at a concentration of 0.5 mg/mL in an ELISA system using immobilized anti-HMGB1 antibody MHa166 (FIG. 10A). Furthermore, effects of the coexisting HMGB2 peptide on HMGB2 measurements were similarly examined using 1,820 ng/mL HMGB2 (FIG. 10B).

As a result, in ELISA using No. 12 polyclonal antibody, the HMGB2 peptide HM2-1 did not affect the measurements of both HMGB1 and HMGB2; however, in the HMGB2 peptide HM2-2-added group, the reaction with HMGB1 was enhanced approximately 1.6-fold, whereas the reaction with HMGB2 was attenuated, and the nonspecific reactions were suppressed (FIG. 10).

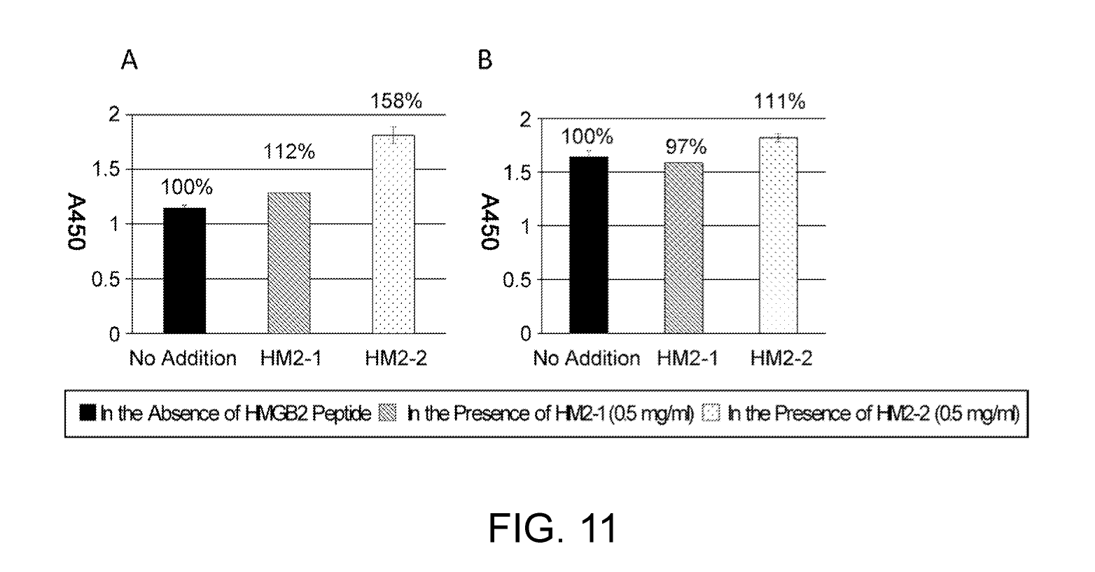

Furthermore, when No. 14 polyclonal antibody was used, the HMGB2 peptide HM2-1 did not have any influence on HMGB1 measurements, but the HMGB2 peptide HM2-2 increased the reactivity of HMGB1 (FIG. 11A). However, neutralizing effects on HMGB2 were not observed for either of the peptides (FIG. 11B).

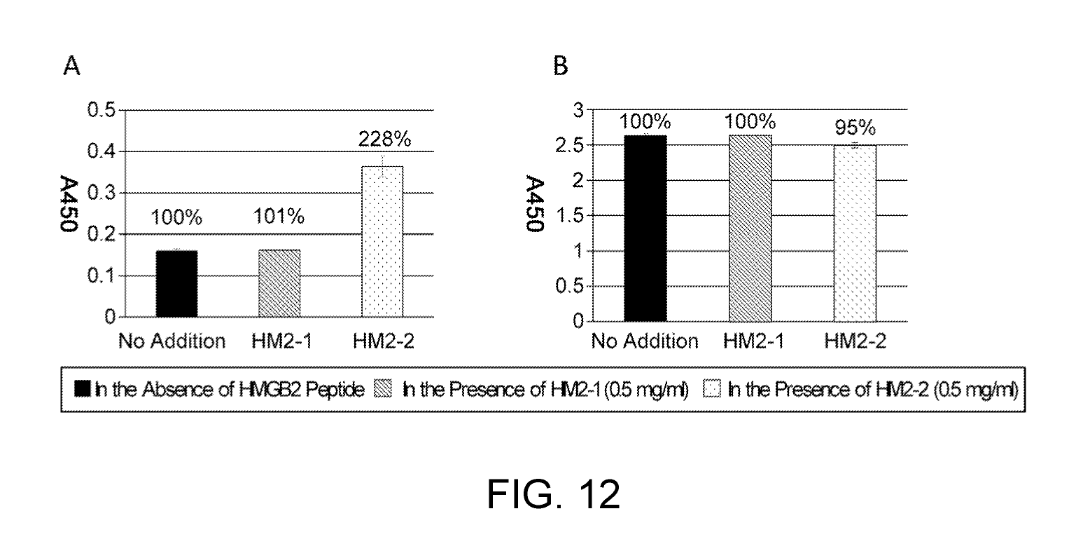

Similar examinations were carried out for systems using another monoclonal antibody. It was examined how the reactions achieved by 50 ng/mL HMGB1 change when MHa176 antibody, which was obtained by immunization with HMGB1, were HRP-labeled and an HMGB2 peptide was allowed to coexist at a concentration of 0.5 mg/mL in an ELISA system with the immobilized anti-HMGB1 antibody MHa166 (FIG. 12A). Furthermore, effects of the coexisting HMGB2 peptide on HMGB2 measurements were similarly examined using 1,820 ng/mL HMGB2 (FIG. 12B).

As a result, in ELISA using the HMa176 antibody, only the enhancement of reactivity of HMGB1 due to the HMGB2 peptide HM2-2 could be observed (FIG. 12).

### [Reference Example] Method of Producing the Antibodies Used in Example 1

### 1. Method of Producing Monoclonal Antibody CP11-1

A hapten-carrier protein complex was prepared by adding a cysteine (C) to the C terminus of the peptide of positions 167 to 180 (KPDAAKKGVVKAEK/SEQ ID NO: 21) in the amino acid sequence of human HMGB1, which was used as a hapten, and linking it via the SH group of the cysteine to keyhole limpet hemocyanin (KLH) which is a carrier. Initial immunization of mice (Balb/c) was carried out using 100 μg of the prepared hapten-carrier protein complex antigen together with Freund's complete adjuvant (FCA), and 14 days later, as a booster, the mice were immunized with 50 μg of the hapten-carrier protein complex, which was used for the initial immunization, together with Freund's incomplete adjuvant (FIA). Thereafter, with 14-day intervals, immunization was carried out four times in total using 50 μg of the hapten-carrier protein complex with FIA. After the final immunization, the spleen was removed, and lymphocytes were prepared according to a standard method and fused with myeloma Sp2/0-Ag14 (ATCC: CRL-1581). The hybridomas resulting from the fusion were cultured and cloned using a ClonaCell-HY Hybridoma Cloning Kit (STEM CELL TECHNOLOGIES). The obtained clones were examined in further detail using Biacore (GE Healthcare), and CP11-1 was selected.

Monoclonal antibody CP11-1 has been deposited as the accession number “NITE P-02020” with the Patent Microorganisms Depositary (NPMD) of the National Institute of Technology and Evaluation.

### 2. Method of Producing Monoclonal Antibodies HMa166 and HMa176

Initial immunization of mice (Balb/c) was carried out using 100 μg of rhHMGB1 antigen together with Freund's complete adjuvant (FCA). Fourteen days later, as a booster, the mice were immunized with 50 μg of the rhHMGB1 antigen together with Freund's incomplete adjuvant (FIA). Thereafter, with 14-day intervals, immunization was carried out four times in total using 50 μg of rhHMGB1 antigen with FIA. After the final immunization, the spleen was removed, and lymphocytes were prepared according to a standard method and fused with myeloma Sp2/0-Ag14 (ATCC: CRL-1581). The hybridomas resulting from the fusion were cultured and cloned by a limiting dilution method according to a standard method. The obtained clones were examined in further detail using Biacore (GE Healthcare), and HMa166 and HMa176 were selected.

Monoclonal antibody HMa166 has been deposited as the accession number “NITE P-02021” with the Patent Microorganisms Depositary (NPMD) of the National Institute of Technology and Evaluation.

### 3. Screening Method for Monoclonal Antibody Production

Screening was carried out as follows. Culture supernatants of each clone were added to an ELISA plate which was prepared by immobilizing 50 μL of rhHMHB1 (2 μg/mL) by a standard method and then blocking it using ImmunoBlock (DS Pharma Biomedical) diluted five-fold in purified water, this was incubated for two hours and then washed. Subsequently, HRP-labeled anti-mouse IgG was added as a secondary antibody, and the mixture was incubated for two hours and then washed. Chromogenic substrate TMBZ (KPL) was added according to a standard method, the reaction was stopped using phosphoric acid, and the absorbance at 450 nm was measured using a microplate reader.

### 4. Method of Producing No. 12 and No. 14 Polyclonal Antibodies

No. 12 (FPAbH12) polyclonal antibody was prepared as follows. An antigen was prepared by adding a cysteine (C) to the C terminus of the peptide of positions 167 to 180 (KPDAAKKGVVKAEK/SEQ ID NO: 21) in the amino acid sequence of human HMGB1, which is a hapten, and linking it via the SH group of the cysteine to keyhole limpet hemocyanin (KLH) which is the carrier. Initial immunization of rabbit (the species New Zealand White) was carried out by using 0.3 mg of the prepared antigen together with Freund's complete adjuvant (FCA). Ten days later, as a booster, the rabbit was immunized with 0.05 mg of recombinant human HMGB1 (rhHMGB1) together with Freund's incomplete adjuvant (FIA) for immunization. Thereafter, with ten-day intervals, immunization was carried out five times in total using 0.05 mg of rhHMGB1 with FIA. After the final immunization, whole blood was collected, purified using a Protein G column, and then subjected to the experiments.

No. 14 (FPAbH14) polyclonal antibody was prepared as follows. Initial immunization of rabbit (the species New Zealand White) was carried out by using 0.1 mg of rhHMGB1 together with FCA. Ten days later, as a booster, the rabbit was immunized with 0.05 mg of rhHMGB1 together with FIA. Thereafter, with ten-day intervals, immunization was carried out five times in total using 0.05 mg of rhHMGB1 with FIA. After the final immunization, whole blood was collected, purified using a Protein G column, and then subjected to the experiments.

### INDUSTRIAL APPLICABILITY

Methods of accurately measuring or detecting HMGB1 alone in samples, and kits and reagents for accurately measuring or detecting HMGB1 alone in samples are provided by the present invention. The methods, kits, and reagents of the present invention are useful for selective measurement or detection of HMGB1 in samples. Furthermore, the methods, kits, and reagents of the present invention are useful as diagnostic markers for sepsis.

1. A method of measuring or detecting HMGB1 in a sample, which comprises the step of contacting an HMGB1 antibody with the sample in the presence of an HMGB2 absorbent.

2. The method of claim 1, wherein the HMGB1 antibody is an antibody having affinity to both HMGB1 and HMGB2.

3. The method of claim 1, wherein the HMGB2 absorbent comprises at least an HMGB2 antibody.

4. The method of claim 1, wherein the HMGB2 absorbent comprises at least a peptide comprising the amino acid sequence of the following formula (I) or (II):
(I): MGKGDPNKPRGKMSSYA (SEQ ID NO: 3); or
(II): CREEHKKKHPDSSVNFAEFS (SEQ ID NO: 4).

5. The method of claim 1, which comprises measuring or detecting HMGB1 by an immunological test method.

6. A kit for measuring or detecting HMGB1, which comprises at least a first reagent comprising an HMGB1 antibody and a second reagent comprising an HMGB2 absorbent.

7. The kit of claim 6, wherein the HMGB1 antibody is an antibody having affinity to both HMGB1 and HMGB2.

8. The kit of claim 6, wherein the HMGB2 absorbent comprises at least an HMGB2 antibody.

9. The kit of claim 6, wherein the HMGB2 absorbent comprises at least a peptide comprising the amino acid sequence of the following formula (I) or (II):
(I): MGKGDPNKPRGKMSSYA (SEQ ID NO: 3); or
(II): CREEHKKKHPDSSVNFAEFS (SEQ ID NO: 4).

10. The kit of claim 6, which is for use in measuring or detecting HMGB1 using an immunological test method.

11. A reagent for measuring or detecting HMGB1, which comprises at least an HMGB1 antibody and an HMGB2 absorbent.

12. The reagent of claim 11, wherein the HMGB1 antibody is an antibody having affinity to both HMGB1 and HMGB2.

13. The reagent of claim 11, wherein the HMGB2 absorbent comprises at least an HMGB2 antibody.

14. The reagent of claim 11, wherein the HMGB2 absorbent comprises at least a peptide comprising the amino acid sequence of the following formula (I) or (II):
(I): MGKGDPNKPRGKMSSYA (SEQ ID NO: 3); or
(II): CREEHKKKHPDSSVNFAEFS (SEQ ID NO: 4).

15. The reagent of claim 11, which is for use in measuring or detecting HMGB1 using an immunological test method.

16. A peptide comprising the amino acid sequence of the following formula (I) or (II):
(I): MGKGDPNKPRGKMSSYA (SEQ ID NO: 3); or
(II): CREEHKKKHPDSSVNFAEFS (SEQ ID NO: 4),
wherein the peptide inhibits binding between HMGB2 and an HMGB1 antibody.

17. A sample which comprises the peptide of claim 16.


SEQUENCE LISTING (21 sequences)

SEQ ID NO: 1 - protein, 215 aa, Homo sapiens [sequence omitted]

SEQ ID NO: 2 - protein, 209 aa, Homo sapiens
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 3 - protein, 17 aa, Artificial sequence
  An artificially synthesized peptide sequence
MGKGDPNKPRGKMSSYA

SEQ ID NO: 4 - protein, 20 aa, Artificial sequence
  An artificially synthesized peptide sequence
CREEHKKKHPDSSVNFAEFS

SEQ ID NO: 5 - protein, 17 aa, Artificial sequence
  An artificially synthesized peptide sequence
MGKGDPKKPRGKMSSYA

SEQ ID NO: 6 - protein, 20 aa, Artificial sequence
  An artificially synthesized peptide sequence
CREEHKKKHPDASVNFSEFS

SEQ ID NO: 7 - protein, 215 aa, Mus musculus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEEEEDEDEEEDDDDE

SEQ ID NO: 8 - protein, 215 aa, Rattus norvegicus; identical to SEQ ID NO: 7

SEQ ID NO: 9 - protein, 215 aa, Oryctolagus cuniculus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 10 - protein, 215 aa, Canis lupus familiaris; identical to SEQ ID NO: 9

SEQ ID NO: 11 - protein, 215 aa, Felis catus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEEEEEDDDDE

SEQ ID NO: 12 - protein, 215 aa, Bos taurus; identical to SEQ ID NO: 11

SEQ ID NO: 13 - protein, 215 aa, Equus caballus; identical to SEQ ID NO: 9

SEQ ID NO: 14 - protein, 215 aa, Sus scrofa
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKHPYEKKAAKLKEKYEKDIAAYRAKGKPDAAKKGVVKAEKSKKKKEEEEDEEDEEDEEEEEDEEDEEEEEDDDDE

SEQ ID NO: 15 - protein, 215 aa, Capra hircus; identical to SEQ ID NO: 11

SEQ ID NO: 16 - protein, 215 aa, Macaca mulatta; identical to SEQ ID NO: 9

SEQ ID NO: 17 - protein, 215 aa, Macaca fascicularis; identical to SEQ ID NO: 9

SEQ ID NO: 18 - protein, 215 aa, Pan troglodytes
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEDEEDEDEEEDDDDE

SEQ ID NO: 19 - protein, 215 aa, Gallus gallus
MGKGDPKKPRGKMSSYAFFVQTCREEHKKKHPDASVNFSEFSKKCSERWKTMSSKEKGKFEDMAKADKLRYEKEMKNYVPPKGETKKKFKDPNAPKRPPSAFFLFCSEFRPKIKGEHPGLSIGDVAKKLGEMWNNTAADDKQPYEKKAAKLKEKYEKDIAAYRAKGKVDAGKKVVAKAEKSKKKKEEEEDEDEDEEDEEDEEEEEEEEEDDDDDE

SEQ ID NO: 20 - protein, 205 aa, Danio rerio
MGKDPTKPRGKMSSYAYFVQTCREEHKKKHPEATVNFSEFSKKCSERWKTMSAKEKGKFEDMAKLDKARYEREMKNYIPPKGEKKKRFKDPNAPKRPPSAFFIFCSEFRPKVKEETPGLSIGDVAKRLGEMWNKISSEEKQPYEKKAAKLKEKYEKDIAAYRSKGKVGGGAAKAPSKPDKANDEDEDDDEEEDEDDDDEEEEDDE

SEQ ID NO: 21 - protein, 14 aa, Artificial Sequence
  An artificially synthesized peptide sequence
KPDAAKKGVVKAEK